(19) United States
(12) Patent Application Publication
(10) Pub. No.: US 2008/0038773 A1
(43) Pub. Date: Feb. 14, 2008

(54) COMPOSITION, METHOD AND KIT FOR REDUCING BACKGROUND STAINING

(75) Inventors: Brian Filanoski, Spokane Valley, WA (US); I. Lawrence Greenfield, Eugene, OR (US); James David Hirsch, Springfield, OR (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/873,281

(22) Filed: Oct. 16, 2007

Related U.S. Application Data
(63) Continuation of application No. 11/054,758, filed on Feb. 9, 2005.

(51) Int. Cl. G01N 1/30; C12Q 1/00

(52) U.S. Cl. 435/40.5; 435/4

(57) ABSTRACT

Compositions, methods and kits are disclosed for improved staining of a cell or tissue with a dye-conjugate that binds specifically to a particular component of the cell or tissue. The compositions, methods and kits include a polymeric material that reduces non-specific binding of a dye-conjugate to components of the cell or tissue other than the particular component specifically bound by the dye-conjugate. In some embodiments, the polymeric material is a synthetic polymer or a naturally-occurring polymer that is substituted by multiple sulfate, sulfonate, phosphate and/or phosphonate groups.

### CROSS-REFERENCE TO RELATED APPLICATIONS

This application claims priority of U.S. Ser. No. 60/543,643, filed Feb. 9, 2004, which disclosure is herein incorporated by reference.

### FIELD OF THE INVENTION

The disclosure relates to methods and compositions that alleviate non-specific background staining of cells and tissues by dye-conjugates. In particular, the disclosure concerns methods and compositions that reduce background staining, such as nuclear background staining, by fluorescent dye-conjugates. The disclosure has applications in the fields of molecular biology, cell biology, immunohistochemistry, diagnostics and therapeutics.

2. BACKGROUND OF THE INVENTION

The ability to label (stain) particular features in cells and tissues using specific reactions, such as immunohistochemical reactions, is important for elucidating cell function and structure. The level of staining (signal) of the specifically-targeted features should be detectable above the background (noise), and should be distinguishable from non-specific staining of other cellular or tissue features. For immunohistochemical techniques, purification of antibody preparations (primary and/or secondary) to maximize specific staining is only a partial solution to the problem of non-specific background staining.

U.S. Pat. No. 4,582,791 (the '791 patent) describes a composition for reducing non-specific background staining in immunofluorescence techniques. The composition includes a combination of a detector conjugate and a non-detector conjugate. The detector conjugate includes a fluorescing moiety bonded to a compound that is capable of specific binding with a material of interest, and the non-detector conjugate includes a poly(amino acid), such as an immunoglobulin, and a mimic compound. The mimic compound of the non-detector conjugate is chosen to have substantial structural and charge similarity to the fluorescing compound of the detector conjugate. The mimic compound also is chosen to exhibit little or no fluorescence, particularly at the wavelengths emitted by the fluorescing moiety of the detector conjugate. The '791 patent teaches that “the components of the non-detector conjugate, e.g., poly(amino-acid) and mimic compound, if employed individually or in combination, in a composition for detecting the presence of a material of interest in a specimen, do not adequately reduce non-specific background fluorescence so that an accurate test may be achieved.” Unfortunately, it can be difficult to identify an appropriate mimic compound for a particular fluorescing moiety, and a different non-detector conjugate must be prepared for each detector-conjugate with a different fluorescing moiety.

A biotin/avidin formulation is disclosed by U.S. Pat. No. 5,487,975 (the '975 patent) that reduces non-specific binding in biotin-avidin detection systems. The formulation includes a biotinylated antibody conjugate, or an avidin-enzyme conjugate, in combination with a diluent. The diluent includes two components; casein (a mixture of proteins derived from milk) and a gamma globulin (the globulin fraction of nonimmune serum). Although the casein and gamma globulin components of this formulation are more defined in composition than the milk and raw serum from which they are derived, they are nonetheless variable mixtures that can provide irreproducible levels of background reduction. Furthermore, the '975 patent does not state that this composition disrupts the non-specific binding of dye-conjugates such as fluorescent dye-conjugates, where charged or hydrophobic dye moieties contribute to non-specific staining.

An additional potential drawback of the background-reducing compositions of the '791 and '975 patents is that they require at least one polypeptide component other than an antibody or avidin. Polypeptides, and especially mixtures of polypeptides such as casein (a mixture of phosphorylated polypeptides) and gamma globulin, can include peptide sequence epitopes that cross-react with and bind to specific-binding conjugates such as antibody conjugates, thereby reducing the level of desired, specific staining of cell or tissue features.

A background-reducing composition that is more defined in its components than casein and gamma globulin, and that can reduce or eliminate non-specific binding of dye-conjugates without the need for choosing a mimic compound and the need to provide a conjugate thereof, would be helpful to overcome the disadvantages of prior background-reducing compositions. Provided herein are novel compositions, methods and kits for reducing or eliminating non-specific binding of a wide range of dye-conjugates in cell and tissue samples.

### SUMMARY OF THE INVENTION

A composition and method are provided for reducing or eliminating non-specific background staining of cells and tissues by dye-conjugates. The composition and method can be used to reduce non-specific background staining of cells and tissues by fluorescent dye-conjugates, for example, fluorescent dye-conjugates of antibodies and other specific-binding agents such as avidin and streptavidin.

The disclosed composition is a blocking solution that includes a polymeric material. The polymeric material includes a polymer having multiple sulfonate, sulfate, carboxylate, phosphate or phosphonate groups or a combination of two or more such polymeric materials. In some embodiments, when the polymeric material includes a poly(amino acid), the poly(amino acid) includes a sulfated, sulfonated or phosphonated poly(amino acid). In other embodiments, when the polymeric material includes a poly(amino acid), the poly(amino acid) is other than casein, IgG, or albumin. In particular embodiments, the polymeric material comprises a synthetic polymer, a polysaccharide or a derivative thereof. In other particular embodiments, the polymeric material of the composition includes a mixture of one or more synthetic polymers and one or more polysaccharides. The composition further includes a material that is useful for composition formulation, for example, a solvent such as water or a buffer. In addition, the blocking solution optionally further includes one or more of a dye-conjugate, a detergent and/or a preservative. The disclosed method for reducing background staining by a dye-conjugate includes contacting a cell or tissue with a dye-conjugate and contacting the cell or tissue with a disclosed background-reducing polymeric material. The cell or tissue can be contacted with the dye-conjugate and the polymeric material in any order, but in particular embodiments, the cell or tissue is first contacted with the polymeric material and then contacted with the dye-conjugate.

Kits also are provided that include the disclosed background-reducing polymeric materials. The polymeric material may be provided in the kit as a solid or in a blocking solution, for example, a buffered blocking solution. Kits can further include a mounting medium or a dye-conjugate, for example, a dye-conjugate dissolved in a blocking solution, dissolved in a separate solution, or as a solid. Such kits can further include instructions for performing the disclosed methods.

### BRIEF DESCRIPTION OF THE DRAWINGS

FIG. 1A is an exemplary fluorescence photomicrograph of bovine pulmonary artery endothelial cells treated with a streptavidin dye-conjugate showing background staining, especially of the nuclei of the cells, in the absence of a dye background-reducing composition.

FIG. 1B is an exemplary fluorescence photomicrograph of bovine pulmonary artery endothelial cells treated with a streptavidin dye-conjugate showing the reduction of background staining in the presence of a disclosed dye background-reducing composition.

FIG. 2A is an exemplary fluorescence photomicrograph of bovine pulmonary artery endothelial cells treated with a streptavidin dye-conjugate showing background staining, especially of the nuclei of the cells, in the absence of a dye background-reducing composition.

FIG. 2B is an exemplary fluorescence photomicrograph of bovine pulmonary artery endothelial cells treated with a streptavidin dye-conjugate showing the reduction of background staining in the presence of a disclosed dye background-reducing composition.

FIG. 3A is an exemplary fluorescence photomicrograph of bovine pulmonary artery endothelial cells treated with a streptavidin dye-conjugate showing background staining, especially of the nuclei of the cells, in the absence of a dye background-reducing composition.

FIG. 3B of is an exemplary fluorescence photomicrograph of bovine pulmonary artery endothelial cells treated with a streptavidin dye-conjugate showing the reduction of background staining in the presence of a disclosed dye background-reducing composition.

FIG. 4A is an exemplary photomicrograph of HeLa cells treated with a mouse monoclonal anti-golgin primary antibody and a fluorescent goat anti-mouse IgG secondary antibody, showing background staining in the absence of a dye background-reducing composition.

FIG. 4B is an exemplary fluorescence photomicrograph of HeLa cells treated with a mouse monoclonal anti-golgin primary antibody and a fluorescent goat anti-mouse IgG secondary antibody, showing the reduction of background staining in the presence of a disclosed dye background-reducing composition.

FIG. 5A is an exemplary fluorescence photomicrograph of perfused and frozen mouse brain tissue treated with a monoclonal mouse anti-Hu C/D primary antibody and a fluorescent goat anti-mouse IgG secondary antibody, showing strong non-specific background fluorescent staining of white matter regions in addition to specific fluorescent staining of the Hu C/D target.

FIG. 5B is an exemplary fluorescence photomicrograph of perfused and frozen mouse brain tissue treated with a monoclonal mouse anti-Hu C/D primary antibody and a fluorescent goat anti-mouse IgG secondary antibody, showing reduction of background staining to autofluorescence levels upon addition of a disclosed dye background-reducing composition.

FIG. 6 is a bar graph showing relative nuclear background fluorescence emitted by untreated samples and by samples treated with disclosed blocking solutions.

FIG. 7 is a bar graph showing relative white matter background fluorescence emitted by untreated samples and by samples treated with disclosed blocking solutions.

FIG. 8 is an exemplary surface plasmon resonance spectra showing non-specific binding of a streptavidin-dye-conjugate to a myelin basic protein-coated sensor surface. The solid line represents non-specific binding in the absence of a dye background-reducing composition. The dotted line represents non-specific binding after pre-treatment of the sensor surface with a dye background-reducing composition. Each line represents the average surface plasmon resonance signal from three different sample applications.

### DETAILED DESCRIPTION OF THE INVENTION

The disclosed compositions, methods and kits will now be further and specifically described.

### Definitions

In order to facilitate an understanding of the embodiments presented, the following abbreviations, terms, explanations and examples are provided. Although methods and materials similar or equivalent to those described herein can be used in practice, suitable methods and materials are described below. The specifically described materials, methods, and examples are illustrative only and not intended to be limiting.

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a dye-conjugate” includes a plurality of conjugates and reference to “a polymeric material” includes a plurality of materials and the like.

Unless defined otherwise, all technical and scientific terms used herein have the same meaning as commonly understood by one of ordinary skill in the art to which this invention is related. The following terms are defined for purposes of the invention as described herein.

The term “blocking solution” as used herein refers to a solution that reduces or eliminates non-specific background staining by a dye-conjugate.

The term “buffer” as used herein refers to a system that acts to minimize the change in acidity or basicity of the solution against addition or depletion of chemical substances.

The term “dye-conjugate” as used herein refers to a molecule that includes a specific binding moiety covalently bonded to a dye moiety, either directly or through a linker, L. The dye-conjugates are added to a sample wherein the specific binding moiety will bind a specific ligand (if present) and be detectable (visually or electronically) when the dye moiety is illuminated with an appropriate light source.

The term “dye moiety” as used herein refers to a compound that emits light to produce an observable detectable signal. Typically, this includes any chemical moiety that exhibits an absorption maximum beyond 280 nm. “Dye” includes without limitations, fluorophores, chromophores, fluorescent proteins, tandem dyes (energy transfer pair), chemiluminescent compounds, and luminescent compounds. The term “fluorophore” as used herein refers to a composition that is inherently fluorescent or demonstrates a change in fluorescence upon binding to a biological compound or metal ion, or metabolism by an enzyme, i.e., fluorogenic. Fluorophores may be substituted to alter the solubility, spectral properties or physical properties of the fluorophore. Numerous fluorophores are known to those skilled in the art and include, but are not limited to cyanines, carbocyanines, pyrenes, coumarines, benzofurans, quinolines, quinazolinones, indoles, benzazoles, borapolyazaindacenes and xanthenes, with the latter including fluoresceins, rhodamines, rosamines, julolidine xanthenes and rhodols as well as other fluorophores described in RICHARD P. HAUGLAND, MOLECULAR PROBES HANDBOOK OF FLUORESCENT PROBES AND RESEARCH CHEMICALS (9^{th} edition, including the CD-ROM, September 2002).

The term “kit” as used herein refers to a packaged set of related components, typically one or more compounds or compositions.

As used herein, the term “Localize” means to accumulate in, or be restricted to, a specific or limited area.

As used herein the term “poly(amino acid)” refers to a polymer of amino acids and/or amino acid analogs and includes peptides, polypeptides and proteins. Amino acids in a poly(amino acid) can be joined with peptide bonds or peptide bond analogs.

The term “reactive group” as used herein refers to a group that is capable of reacting with another species, such as an atom or chemical group(s) to form a covalent bond, and includes nucleophiles, electrophiles and photoactivatable groups. Thus, a reactive dye is a dye moiety that comprises a reactive group. In this way the reactive group on the dye functions as the site of attachment for another moiety, such as a specific binding moiety to form a dye-conjugate.

The term “target” as used herein refers to any species that is being identified or quantified by a specific binding reaction, also referred to as a ligand. A target can, for example, be a protein, a peptide, a nucleic acid, an oligonucleotide, a carbohydrate, a polysaccharide or small molecule. A protein can, for example, be an enzyme, a nucleic acid binding protein, an organelle-associated protein, a cytoplasmic protein or a regulatory protein.

The term “specific binding moiety”, as used herein, refers to species that will selectively localize a target in a particular tissue or cell, or a region of a tissue or a cell, e.g., a ligand receptor. Localization of the specific binding moiety within a tissue or a cell can be mediated, for example, by specific recognition of molecular determinants, the molecular size of the targeting agent or conjugate, ionic interactions or hydrophobic interactions or various combinations of these features. Other mechanisms of targeting an agent to a particular tissue or region are known to those of skill in the art. Exemplary specific binding moieties include antibodies, antibody fragments, transferrin, HS-glycoprotein, coagulation factors, serum proteins, β-glycoprotein, G-CSF, GM-CSF, M-CSF, EPO and the like. In some embodiments, a specific binding moiety binds specifically to another substance, for example, a protein, a nucleic acid, a cell or a cell component, such as an organelle. As used herein, a specific binding moiety such as an antibody, or antigen-binding fragment thereof, is said to “bind specifically” if it reacts at a detectable level (within, for example, an ELISA) with a substance, such as a protein, and does not react detectably with unrelated substances, such as other peptides, polypeptides, nucleic acids and proteins, under similar conditions. As used herein, “binding” refers to a noncovalent association between two separate molecules such that a complex is formed. The ability to bind may be evaluated by, for example, determining a binding constant for the formation of the complex. The binding constant is the value obtained when the concentration of the complex is divided by the product of the component concentrations. In particular embodiments, two compounds are said to “bind specifically” when the binding constant for complex formation exceeds about 10² L/mol, for example, exceeds 10³ L/mol, or exceeds 10⁴ L/mol, or greater. The binding constant may be determined using methods well known in the art.

It is further to be understood that all molecular weight or molecular mass values given for compounds are approximate, and are provided for description only. Although methods and materials similar or equivalent to those described herein can be used in the practice or testing of this disclosure, suitable methods and materials are described below. Thus, the specific materials, methods, and examples provided below are illustrative only and not intended to be limiting.

In general, for ease of understanding the present invention, the blocking solution and corresponding polymeric materials will first be described in detail, followed by the dye-conjugates, the methods in which the blocking solution and dye-conjugates find uses, which is followed by exemplified methods of use and kits.

Blocking Solution

The disclosed compositions include a blocking solution for reducing non-specific background staining of cells and tissue sections by dye-conjugates, wherein a dye-conjugate comprises a dye-moiety and a specific binding partner. The present blocking solution is particularly efficient at reducing background staining wherein the dye moiety participates in the non-specific staining of a sample, these dye moieties include moieties that contain a sulfo moiety, carboxy moiety and/or a fluorine atom. In some embodiments, the blocking solution includes a polymeric material bearing multiple carboxylate, sulfate, sulfonate, phosphate and/or phosphonate groups and a solvent such as a buffer. The blocking solution can be added before, during or after the addition of the desired dye-conjugate, thereby resulting in a better-resolved view of a desired target of the dye-conjugate. The blocking solution is particularly advantageous for reducing background staining in cell nuclei and neuronal white matter. In addition, the blocking solution may be used either together with or in place of traditional background reducing treatments, such as treatments of a cell or tissue with an albumin such as bovine serum albumin (BSA).

i. Polymeric Materials

The polymeric materials typically possess one, two, three or more negative charges when dissolved in a solvent. If the polymeric material includes a poly(amino acid), the polymeric material can include a sulfated, sulfonated or phosphonated poly(amino acid). In some embodiments, the polymeric material is other than sulfobutylether β-cyclodextrin, casein, IgG or albumin. Typically, the polymeric material exhibits little or no fluorescence, particularly at the wavelengths emitted by fluorescent dye-conjugates.

In some embodiments, the polymeric material includes a synthetic polymer, a carbohydrate polymer or a sulfated poly(amino acid), or a combination or mixture thereof. In particular embodiments, the polymeric material includes a sulfated or sulfonated carbohydrate. In other particular embodiments, the polymeric material includes a polystryrene or copolymer thereof; a polyacrylamide or copolymer thereof; a polyvinylene or copolymer thereof; a polyacrylate or copolymer thereof; a polyalkalene or copolymer thereof; a polyaniline or copolymer thereof; a polyphenylalkylene of copolymer thereof; a glycosaminoglycan or derivative thereof; a heparin or derivative thereof; a dextran or derivative thereof; a suramin or derivative thereof; carrageenan or derivative thereof; a cyclodextrin other than sulfobutylether beta-cyclodextrin or derivative thereof; a cellulose or derivative thereof; a pentosan or derivative thereof; a dextrin or derivative thereof; a laminarin or derivative thereof; a dermatan or derivative thereof; a chitin or derivative thereof; a chitosan or derivative thereof; a curdlan or derivative thereof; a pullulan or derivative thereof; a keratan or derivative thereof; a fucoidan or derivative thereof; a ficoll or derivative thereof, a xylan or derivative thereof; an amylose or derivative thereof; a galactan or derivative thereof; a mucin or derivative thereof; a galactomannan or derivative thereof; a mannan or derivative thereof; a glucan or derivative thereof; a fucan or derivative thereof; a heparaosan or derivative thereof; a rhamnan or derivative thereof; a catechin or derivative thereof; or, a calixarene or derivative thereof, or a combination or mixture thereof.

In more particular embodiments, the polymeric material includes poly(sodium 4-styrenesulfonic acid), poly(4-styrenesulfonic acid-co-maleic acid), poly(2-acrylamido-2-methyl-1-propanesulfonic acid), poly(vinylsulfate), poly(vinylsulfonic acid), poly(vinylphosphate), poly(vinylphosphonic acid), poly(anilinesulfonic acid), poly(anetholesulfonic acid), heparin, heparin-like substance, deaminated heparin, chondroitin sulfate, dextran sulfate, sulfopropyl-beta-cyclodextrin, beta-cyclodextrin tetradecasulfate, poly(1-tetradecene-sulfone), poly(ethyleneglycol)-4-nonylphenyl-3-sulfopropyl ether, suramin, poly(propenesulfate), poly(butenesulfate), poly(pentanesulfate), poly(hexenesulfate), poly(heptenesulfate), poly(octenesulfate), poly(nonenesulfate), poly(decenesulfate), poly(undecenesulfate), poly(dodecenesulfate), poly(phenylnonenesulfate), poly(phenyldecenesulfate), poly(phenylundecenesulfate), poly(phenyldodecenesulfate), poly(styrenesulfate), poly(vinylnaphthalenesulfate), poly(vinylbiphenylsulfate), poly(sulfatephenylpropene), poly(sulfatephenylbutene), poly(sulfatephenylpentene), poly(sulfatephenylhexene), poly(sulfatephenylheptene), poly(sulfatephenyloctene), poly(sulfatephenylnonene), poly(sulfatephenyldecene), poly(sulfatephenylundecene), poly(sulfatephenyldodecene), poly(propenesulfonate), poly(butenesulfonate), poly(pentenesulfonate), poly(hexenesulfonate), poly(heptenesulfonate), poly(octenesulfonate), poly(nonenesulfonate), poly(decenesulfonate), poly(undecenesulfonate), poly(dodecenesulfonate), poly(vinylnaphthalenesulfonate), poly(vinylbiphenylsulfonate), sulfonated poly(vinylphenylketone), sulfonated poly(phenylsulfone), sulfonated poly(4-methylstyrene), sulfonated poly(alpha-methylstyrene), sulfonated poly(styrene-block-ethyleneoxide-block-styrene), sulfonated poly(ethyleneoxide-block-styrene-block-ethyleneoxide), sulfonated poly(4-methoxystyrene), sulfonated poly(ethyleneoxide-block-styrene), sulfonated poly(styrene-block-ethylene), sulfonated poly(acenaphthylene), sulfonated poly(vinylcarbazole), sulfonated poly(styrene-co-butadiene), sulfonated poly(styrene-block-(ethylene-co-butylene)-block-styrene, poly(naphthalene-2-sulfonate), poly(methylenehydroquinonesulfonate), poly(styrenesulfonate-co-styrene), poly(styrenesulfonate-co-acrylic acid), poly(styrenesulfonate-co-methacrylic acid), poly(styrenesulfonate-co-acrylamidomethylpropanesulfonate), poly(styrenesulfonate-co-itaconic acid), poly(styrenesulfonate-co-vinylbenzoic acid), poly(styrenesulfonate-co-octylstyrenesulfonamide), polystyrenesulfonate-co-menthylstyrenesulfonate), poly(styrenesulfonate-co-lithocholic acid styrenesulfonate), poly(styrenesulfonate-co-diallylmethylammonium chloride), poly(styrenesulfonate-co-diallyidimethylammonium chloride), poly(styrenesulfonate-co-diallylmethyloctylammonium chloride), poly(styrenesulfonate-co-allylamine), poly(styrenesulfonate-co-vinylamine), poly(styrenesulfonate-co-vinylbenzyltrimethylammonium chloride), poly(sulfophenethylacrylamide), poly(sulfophenethylmethacrylamide), poly(sulfophenylpropene), poly(sulfophenylbutene), poly(sulfophenylpentene), poly(sulfophenylhexene), poly(sulfophenylheptene), poly(sulfophenyloctene), poly(sulfophenylnonene), poly(sulfophenyldecene), poly(sulfophenylundecene), poly(sulfophenyldodecene), poly(styrenesulfanilate), poly(propenephosphate), poly(butenephosphate), poly(pentenephosphate), poly(hexenephosphate), poly(heptenephosphate), poly(octenephosphate), poly(nonenephosphate), poly(decenephosphate), poly(undecenephosphate), poly(dodecenephosphate), poly(propenephosphonate), poly(butenephosphonate), poly(pentenephosphonate), poly(hexenephosphonate), poly(heptenephosphonate), poly(octenephosphonate), poly(nonenephosphonate), poly(decenephosphonate), poly(undecenephosphonate), polydodecenephosphonate), poly(phosphophenylpropene), poly(phosphophenylbutene), poly(phosphophenylpentene), poly(phosphophenylhexene), poly(phosphophenylheptene), poly(phosphophenyloctene), poly(phosphophenylnonene), poly(phosphophenyldecene), poly(phosphophenylundecene), poly(phosphophenyldodecene), poly(phosphatephenylpropene), poly(phosphatephenylbutene), poly(phosphatephenylpentene), poly(phosphatephenylhexene), poly(phosphatephenylheptene), poly(phosphate phenyloctene), poly(phosphate phenylnonene), poly(phosphatephenyldecene), poly(phosphatephenylundecene), poly(phosphatephenyldodecene), poly(diphenoxyphosphazene), carrageenan, pentosan sulfate, pentosan phosphate, pentosan phophosulfate, cellulose sulfate, cellulose phosphate, cellulose phophosulfate, dextrin sulfate, dextrin phosphate, dextrin phosphosulfate, laminarin sulfate, laminarin phosphate, laminarin phosphosulfate, dermatan sulfate, dermatan phosphate, dermatan phosphosulfate, chitin sulfate, chitin phosphate, chitin phosphosulfate, chitosan sulfate, chitosan phosphate, chitosan phosphosulfate, curdlan sulfate, curdlan phosphate, curdlan phosophosulfate, pullulan sulfate, pullulan phosphate, pullulan phosphosulfate, hyaluronic acid sulfate, hyaluronic acid phosphate, hyaluronic acid phosphosulfate, keratan sulfate, keratan phosphate, keratan phosphosulfate, fucoidan sulfate, fucoidan phosphate, fucoidan phosphosulfate, ficoll sulfate, ficoll phosphate, ficoll phosphosulfate, xylan sulfate, xylan phosphate, xylan phosphosulfate, amylose sulfate, amylose phosphate, amylose phosphosulfate, D-galactan sulfate, D-galactan phosphate, D-galactan phosphosulfate, N-(carboxymethyl)chitosan sulfate, N-(carboxymethyl)chitosan phosphate, N-(carboxymethyl)chitosan phosphosulfate, mucin sulfate, mucin phosphate, mucin phosphosulfate, galactomannan sulfate, galactomannan phosphate, galactomannan phosphosulfate, mannan sulfate, mannan phosphate, mannan phosphosulfate, glucan sulfate, glucan phosphate, glucan phosphosulfate, fucan sulfate, fucan phosphate, fucan phosphosulfate, N-acetylheparosan sulfate, N-acetylheparosan phosphate, N-acetylheparosan phosphosulfate, rhamnan sulfate, rhamnan phosphate, rhamnan phosphosulfate, (−)-epicatechin sulfate, 4-sulfocalix[4]arene, 4-sulfonatocalix[8]arene, sulfated insulin, polymeric sulfated IgA, polymeric sulfated IgD, polymeric sulfated IgE, polymeric sulfated IgG, polymeric sulfated IgM, sulfated silk fibroin, gastrin sulfate, cholecystokinin, polyaspartic acid, polyglutamic acid, poly(tyrosinesulfate), or poly(sulfophenylalanine), or a combination or mixture thereof.

In even more particular embodiments, the polymeric material includes poly(sodium 4-styrenesulfonic acid), poly(4-styrenesulfonic acid-co-maleic acid), poly(2-acrylamido-2-methyl-1-propanesulfonic acid), poly(vinylsulfate), poly(vinylsulfonic acid), poly(vinylphosphate), poly(vinylphosphonic acid), poly(anilinesulfonic acid), poly(anetholesulfonic acid), heparin, heparin-like substance, deaminated heparin, chondroitin sulfate, dextran sulfate, sulfopropyl-beta-cyclodextrin, or beta-cyclodextrin tetradecasulfate, or a combination or mixture thereof.

In most particular embodiments, the polymeric material comprises heparin or dextran sulfate. The heparin can be porcine Type 1-A heparin. The dextran sulfate can be a dextran sulfate having an average molecular weight of from about 5,000 to about 1,000,000, for example, a dextran sulfate having an average molecular weight of about 15,000 to about 100,000.

ii Solvent

The disclosed blocking solution also includes a solvent such as water or a buffer. The polymeric material can be dissolved in the solvent at a concentration that is selected for particular results or applications, such as a concentration from about 0.1 mg/mL to about 20 mg/mL, for example, a concentration from about 0.5 mg/mL to about 10 mg/mL such as a concentration from about 1 mg/mL to about 5 mg/mL.

The buffer solution includes a buffer that does not substantially interfere with a specific binding reaction of a dye-conjugate. The buffer can be the buffer used in the staining procedure used for a dye-conjugate or a different buffer. In some embodiments, the buffer is a “physiological buffer,” which refers to a liquid medium that mimics the salt balance and pH of the cytoplasm of a cell or of the extracellular milieu. Examples of buffers include phosphate buffers (PB), Tris buffers, acetate buffers, bicarbonate buffers, carbonate buffers, borate buffers, citrate buffers, phosphate buffered saline (PBS) buffers and the like. Other examples of buffers include BES, Bicine, EPPS, HEPES, MES, MOPS, MOPSO, PIPES, TAPS, TAPSO, TES, Tricine, Trimethylammonium acetate, ADA, ACES, DIPS, DIPSO, AMPS, AMPSO, CAPS and the like. Particular examples of physiological buffers include phosphate-, Tris- or borate-buffered saline (PBS, TBS or BBS) with pHs ranging from 6.5 to 8.0, or non-saline buffers such as acetates, bicarbonates, or citrates within this pH range. In a particular embodiment, the buffer comprises a PBS buffer, for example, 0.01M phosphate buffer, 0.15M NaCl, at pH 7.2. In general, however, dye background reducing compositions may have any pH between 0 and 14 established by a particular buffer.

iii Optional Components of the Blocking Solution

The disclosed blocking solution optionally includes a detergent, preservative, dye-conjugate or other component that finds use in the blocking solution or for the particular assay being performed.

a. Detergent

The disclosed blocking solution may further include a detergent. The detergent is in an amount sufficient to reduce surface tension of the solution to provide for even sheeting of the buffer to ensure that the experimental sample is effectively covered by the blocking solution. Suitable detergents are those which are compatible with histochemical and cytochemical staining reagents and immunochemical reagents in general and can be any of the nonionic biological detergents used by biochemists for the solubilization of proteins and membrane components. Polyoxyethylenesorbitans and polyoxyethylene ethers are examples of such non-ionic detergents, and specific examples include polyoxyethylenesorbitan monolaurate (sold under the name Tween 20) and polyoxyethylene 23 lauryl ether (sold under the name Brij 35). Both detergents are available from a variety of sources including Sigma-Aldrich (St. Louis, Mo.). The detergent is generally used at a concentration of from about 0.01 to about 5% (v/v), more typically from about 0.05 to about 1% (v/v), such as from about 0.05 to about 0.5% (v/v).

b. Preservative

The blocking solution also may further include a preservative. For example, the composition can include an antimycotic or antimicrobial agent in an effective concentration to inhibit growth of microorganisms in the solution. Preservatives that do not interfere with specific binding reactions, such as immunochemical reactions, are well known. The preservative can be a broad spectrum preservative, such as one that is effective against both bacteria (both gram positive and gram negative) and fungi, or a limited spectrum preservative, such as one that is only effective on a single group of microorganisms. A limited spectrum preservative can be used in combination with a broad spectrum preservative or other limited spectrum preservatives with complimentary and/or supplementary activity. Preservatives are typically effective at concentrations in the range of from about 0.001% to 0.1%, more typically at about 0.01 to about 0.1%, and even more typically at about 0.05%.

Exemplary preservative agents include azides, metal chelating agents, gentamycin, penicillin, streptomycin, thimerosal, and a mixture of 2-chloro-2-methyl-4-isothiazolin-3-one, 2-methyl-4-isothiozolin-3-one and triethylorthoformate in dipropylene glycol, which is available commercially under the product name PROCLIN 300 (Supelco, Inc., Bellefonte, Pa.). In some embodiments, merthiolate (thimerosol) or sodium azide is added, for example, at 0.01 to 0.05%, to retard microbial growth. In some cases where a specific group of microbial contaminants is problematic (such as Gram negatives), aminocarboxylate chelators may be used alone or as potentiators in conjunction with other preservatives. These chelators include, for example, ethylenediaminetetraacetic acid (EDTA), ethylene glycol-bis(2-aminoethyl)-N,N,N′,N″-tetraacetic acid (EGTA, o-phenanthroline hydroxyethylenediaminetriacetic acid, diethylenetriaminepentaacetic acid, and other aminocarboxylate chelators, and mixtures thereof, and their salts, and mixtures thereof.

c. Dye-Conjugate

In other embodiments, the disclosed blocking solution can further include a dye conjugate. In this way the blocking solution and dye conjugates are added simultaneously to the sample. However, it is understood that the dye-conjugate is typically added in a separate step from the step of contacting the sample with the present blocking. Thus, the following section discloses many dye-conjugates that can be used with the present blocking solution; however, the disclosure is not intended to be limiting as many dye-conjugates, a dye moiety covalently bonded to a specific binding partner, can be contemplated that would result in decreased background staining when used in conjunction with the present blocking solution.

The dye moiety of the dye-conjugate can include, without limitation, a chromophore, a fluorophore, a fluorescent protein, or a tandem dye (energy transfer pair). In some embodiments, a dye is any chemical moiety that exhibits an absorption maximum beyond 280 nm.

Examples of dyes include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine or carbocyanine including any corresponding compounds in U.S. Ser. Nos. 09/968,401; 09/557,275 and 09/969,853 and U.S. Pat. Nos. 6,403,807; 6,348,599; 5,486,616; 5,268,486; 5,569,587; 5,569,766; 5,627,027; 6,048,982; 4,981,977; 5,808,044; 5,877,310; 6,002,003; 6,004,536; 6,008,373; 6,043,025; 6,127,134; 6,130,094; and 6,133,445), a carbostyryl, a porphyrin, a salicylate, an anthranilate, an azulene, a perylene, a pyridine, a quinoline, a xanthene (including any corresponding compounds disclosed in U.S. Pat. Nos. 6,162,931; 6,130,101; 6,229,055; 6,339,392; 5,451,343 6,221,606; 6,358,684; 6,008,379; 6,111,116; 6,184,379; 6,017,712; 6,080,852; 5,847,162 and U.S. Ser. No. 09/922,333) an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including any corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs. As is the case for many dyes, they can also function as both chromophores and fluorophores, resulting in dye-conjugates that simultaneously act both as colorimetric and fluorescent labels for target molecules.

In particular embodiments, the dyes include coumarins, xanthenes, pyrenes and cyanines. Typically these dye moieties are anionic or neutral and are substituted by at least one sulfo moiety, carboxy moiety or a fluorine atom and include dyes sold under the tradenames ALEXA FLUOR, OREGON GREEN, CASCADE BLUE, CY DYES, DY and ATTO DYES. Thus, in more particular embodiments the dye-conjugate includes a fluorescent dye, for example, an anionic xanthene, a neutral xanthene, an anionic pyrene, an anionic cyanine, (such as a carbocyanine) a neutral coumarin or an anionic coumarin. In particular embodiments, the dye-conjugate includes a sulfonated fluorescent dye. In an alternative embodiment, the disclosed blocking solution can include a dye-conjugate and a polymeric material, where the dye-conjugate includes a sulfonated fluorescent dye moiety and the polymeric material bears multiple sulfate or sulfonate groups.

Where the dye is a xanthene, the dye is optionally a fluorescein, a rhodol (including any corresponding compounds disclosed in U.S. Pat. Nos. 5,227,487 and 5,442,045), a rosamines or a rhodamine (including any corresponding compounds in U.S. Pat. Nos. 5,798,276 and 5,846,737). As used herein, rhodamine and rhodol dyes include, among other derivatives, compounds that comprise xanthenes with saturated or unsaturated “julolidine” rings. As used herein, fluoresceins include benzo- or dibenzofluoresceins, seminaphthofluoresceins, or naphthofluoresceins. Similarly, as used herein rhodols includes seminaphthorhodafluors (including any corresponding compounds disclosed in U.S. Pat. No. 4,945,171).

In one aspect, the dye moiety has an absorption maximum beyond about 480 nm. In a particularly useful embodiment, the dye absorbs at or near 488 nm to 514 nm (particularly suitable for excitation by the output of the argon-ion laser excitation source) or near 546 nm (particularly suitable for excitation by a mercury arc lamp). In another aspect, the dye absorbs light at wavelengths greater than 546 nm, for example, absorbs light between about 555 nm and 750 nm.

Fluorescent proteins also find use as dye moieties that form dye-conjugates. Examples of fluorescent proteins include green fluorescent protein (GFP), acqurin and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorophore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556 and the sulforhodamine fluorophores disclosed in U.S. Pat. No. 5,798,276, or the sulfonated cyanine fluorophores disclosed in U.S. Ser. Nos. 09/968/401 and 09/969/853; or the sulfonated xanthene derivatives disclosed in U.S. Pat. No. 6,130,101 and those combinations disclosed in U.S. Pat. No. 4,542,104. Alternatively, the fluorophore functions as the energy donor and the fluorescent protein is the energy acceptor. Particularly useful fluorescent proteins are the phycobiliproteins disclosed in U.S. Pat. Nos. 4,520,110; 4,859,582; 5,055,556 (supra) and the fluorophore bilin protein combinations disclosed in U.S. Pat. No. 4,542,104. Alternatively, two or more fluorophore dyes can function as an energy transfer pair wherein one fluorphore is a donor dye and the other is the acceptor dye (including any dye compounds disclosed in U.S. Pat. Nos. 6,358,684; 5,863,727; 6,372,445 and 5,656,554 and those sold under the trade name DyeMer™ dye-conjugate).

The specific binding moiety of the dye-conjugate, as used herein, refers to species that will selectively localize in a particular tissue or cell, or a region of a tissue or a cell by binding to a specific ligand (its specific binding partner). Examples of specific binding moieties include an avidin or binding fragment thereof; a streptavidin or binding fragment thereof; an antibody or a binding fragment thereof; a receptor protein or a binding fragment thereof; a protein A or a binding fragment thereof; a protein G or a binding fragment thereof; a nucleic acid (for example, naturally occurring or synthetic RNA, DNA or cDNA); an enzyme or binding fragment thereof; a metal ion chelator or a metal-chelating fragment thereof; or, a lectin or a binding fragment thereof. These general types of specific binding agents and their targets are summarized in Table 1 below. The dye-conjugate can specifically bind directly with a particular target in a cell or tissue, or can specifically bind to another specific binding molecule that specifically binds to a particular target in a cell or tissue. For example, the dye-conjugate can be a dye-labeled antibody that specifically binds a particular cell or tissue component, or the dye-conjugate can be a dye-labeled antibody that specifically binds to an antibody (such as an IgG) that specifically binds a particular cell or tissue component. Thus, a specific binding partner may be a cellular ligand or it may be a primary antibody or it may be a secondary antibody.

Localization of the specific binding moiety within a tissue or a cell can be mediated, for example, by specific recognition of molecular determinants, molecular size of the targeting agent or conjugate, ionic interactions or hydrophobic interactions or various combinations of these features. Other mechanisms of targeting an agent to a particular tissue or region are known to those of ordinary skill in the art. Exemplary specific binding moieties include antibodies, antibody fragments, transferrin, HS-glycoprotein, coagulation factors, serum proteins, β-glycoprotein, G-CSF, GM-CSF, M-CSF, EPO and the like. In some embodiments, a specific binding moiety binds specifically to another substance, for example, a protein, a nucleic acid, a cell or a cell component, such as an organelle. As used herein, a specific binding moiety such as an antibody, or antigen-binding fragment thereof, is said to “bind specifically” if it reacts at a detectable level (within, for example, an ELISA) with a targeted substance, such as a protein, and does not react detectably with unrelated substances, such as other peptides, polypeptides, nucleic acids and proteins, under similar conditions, or where the level of differential binding is sufficient to discriminate between the presence and absence of the targeted ligand. The ability to bind also may be evaluated by, for example, determining a binding constant for the formation of the complex. The binding constant is the value obtained when the concentration of the complex is divided by the product of the component concentrations. In particular embodiments, two compounds are said to “bind specifically” when the binding constant for complex formation exceeds about 10² L/mol, for example, exceeds 10³ L/mol, or exceeds 10⁴ L/mol. The binding constant may be determined using methods well known in the art.

TABLE 1
Representative Specific Binding Pairs

| Target | Specific binding agent |
|---|---|
| antigen | Antibody |
| Antibody | Antigen |
| Antibody | Antibody |
| Biotin | avidin (or streptavidin or anti-biotin) |
| IgG* | protein A or protein G |
| Drug | drug receptor |
| Drug receptor | Drug |
| Folate | folate binding protein |
| Toxin | toxin receptor |
| carbohydrate | lectin or carbohydrate receptor |
| Lectin or | Carbohydrate |
| carbohydrate | |
| receptor | |
| peptide | peptide receptor |
| protein | protein receptor |
| enzyme substrate | Enzyme |
| Enzyme | Enzyme substrate |
| DNA (RNA) | cDNA (cRNA)† |
| hormone | hormone receptor |
| Ion | chelator |

*IgG is an immunoglobulin
†cDNA and cRNA are the complementary strands used for hybridization

In an exemplary embodiment, the specific binding moiety is an amino acid (including those that are protected or are substituted by phosphates, carbohydrates, or C₁ to C₂₂ carboxylic acids), or a polymer of amino acids such as a peptide or protein. In a related embodiment, the specific binding molecule contains at least five amino acids, for example, 5 to 36 amino acids. Exemplary peptides include, but are not limited to, neuropeptides, cytokines, toxins, protease substrates, and protein kinase substrates. Other exemplary peptides may function as organelle localization peptides, that is, peptides that serve to target the conjugated compound for localization within a particular cellular substructure by cellular transport mechanisms. Such protein-specific binding moieties include enzymes, antibodies, lectins, glycoproteins, histones, albumins, lipoproteins, avidin, streptavidin, protein A, protein G, phycobiliproteins and other fluorescent proteins, hormones, toxins and growth factors. More typically, the protein-specific binding moiety is an antibody, an antibody fragment (such as an antigen-binding fragment, such as a F(ab) fragment, avidin, streptavidin, a toxin, a lectin, or a growth factor. Exemplary haptens include biotin, digoxigenin and fluorophores.

In another exemplary embodiment, the specific binding moiety is a nucleic acid-specific binding moiety such as a nucleic acid base, nucleoside, nucleotide or a nucleic acid polymer, optionally containing an additional linker or spacer for attachment of a fluorophore or other ligand, such as an alkynyl linkage (U.S. Pat. No. 5,047,519), an aminoallyl linkage (U.S. Pat. No. 4,711,955). In another exemplary embodiment, the nucleotide is a nucleoside or a deoxynucleoside or a dideoxynucleoside. Exemplary nucleic acids include single- or multi-stranded, natural or synthetic DNA or RNA oligonucleotides, or DNA/RNA hybrids, or incorporating a linker such as morpholine derivatized phosphates (AntiVirals, Inc., Corvallis Oreg.), or peptide nucleic acids such as N-(2-aminoethyl)-glycine units, where the nucleic acid contains fewer than 50 nucleotides, more typically fewer than 25 nucleotides. The dye moiety of the conjugate is typically attached to nucleic acid-specific binding moieties either directly or through a reactive group to the nucleic acid via one or more purine or pyrimidine bases through an amide, ester, ether or thioether bond; or attached to the phosphate or carbohydrate by a bond that is an ester, thioester, amide, ether or thioether. Alternatively, the compound is attached by formation of a non-covalent association of the nucleic acid and a photoreactive linker of the invention, followed by illumination, resulting in a covalently bound linker.

Alternatively, the specific binding moiety is a cell, a cellular system, a cellular fragment, or a subcellular particle, including a virus particle, a bacterial particle, a virus component, a biological cell (such as an animal cell, plant cell, bacterial cell, or a fungal cell), or a cellular component. Examples of cellular components include lysosomes, endosomes, cytoplasm, nuclei, histones, mitochondria, Golgi apparatus, endoplasmic reticulum, peroxisomes, centrioles and vacuoles.

Dye-conjugates can be purchased from a number of commercial sources (for example, from Invitrogen (Molecular probes detection technologies), Eugene, Oreg.). Dye-conjugates also can be prepared using reactive dyes in well-known methods. As used herein a reactive group includes a nucleophile, an electrophile or photoactivatable group. In some embodiments, a nucleophile and an electrophile react to form a covalent bond (see Table 2). In an exemplary embodiment, the dye moiety includes a reactive group such as an acrylamide, an activated ester of a carboxylic acid, an acyl azide, an acyl nitrile, an aldehyde, an alkyl halide, an anhydride, an aniline, an aryl halide, an azide, an aziridine, a boronate, a carboxylic acid, a diazoalkane, a haloacetamide, a halotriazine, a hydrazine, a hydrazide, an imido ester, an isocyanate, an isothiocyanate, a maleimide or other Michael acceptor, a phosphoramidite, a reactive platinum complex, a succinimidyl ester, a sulfosuccinimidyl ester, a tetrafluorophenyl ester, a sulfonyl halide, a thiol group, or a photoactivatable group. Typically the reactive group present on the dye readily reacts with a protein, peptide or nucleotide.

In particular embodiments, reactive dyes having amine reactive groups are used to form dye-conjugates with antibodies or fragments thereof. In this way, the reactive group of a dye and a reactive group of a specific binding moiety form an electrophile/nucleophile pair that can react to form a covalent linkage between the dye and the specific binding moiety. The electrophile in the pair can be on the dye moiety or on the specific binding moiety. Likewise the nucleophile can be on the dye moiety compound or on the specific binding moiety. Typically, the conjugation reaction between the reactive group on the dye moiety and the reactive group on the specific binding moiety results in one or more atoms of the reactive group being incorporated into a new linkage attaching the dye and specific binding moieties.

TABLE 2
Examples of some routes to useful covalent linkages with
electrophile and nucleophile reactive groups

| Electrophilic Group | Nucleophilic Group | Resulting Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| Aldehydes | amines/anilines | imines |
| aldehydes or ketones | Hydrazines | hydrazones |
| aldehydes or ketones | Hydroxylamines | oximes |
| alkyl halides | amines/anilines | alkyl amines |
| alkyl halides | carboxylic acids | esters |
| alkyl halides | Thiols | thioethers |
| alkyl halides | alcohols/phenols | ethers |
| alkyl sulfonates | Thiols | thioethers |
| alkyl sulfonates | carboxylic acids | esters |
| alkyl sulfonates | alcohols/phenols | ethers |
| Anhydrides | alcohols/phenols | esters |
| Anhydrides | amines/anilines | carboxamides |
| aryl halides | Thiols | thiophenols |
| aryl halides | Amines | aryl amines |
| Aziridines | Thiols | thioethers |
| Boronates | Glycols | boronate esters |
| carboxylic acids | amines/anilines | carboxamides |
| carboxylic acids | Alcohols | esters |
| carboxylic acids | Hydrazines | hydrazides |
| Carbodiimides | carboxylic acids | N-acylureas or anhydrides |
| Diazoalkanes | carboxylic acids | esters |
| Epoxides | Thiols | thioethers |
| Haloacetamides | Thiols | thioethers |
| Halotriazines | amines/anilines | aminotriazines |
| Halotriazines | alcohols/phenols | triazinyl ethers |
| imido esters | amines/anilines | amidines |
| Isocyanates | amines/anilines | ureas |
| Isocyanates | alcohols/phenols | urethanes |
| Isothiocyanates | amines/anilines | thioureas |
| Maleimides | Thiols | thioethers |
| Phosphoramidites | Alcohols | phosphite esters |
| silyl halides | Alcohols | silyl ethers |
| sulfonate esters | amines/anilines | alkyl amines |
| sulfonate esters | Thiols | thioethers |
| sulfonate esters | carboxylic acids | esters |
| sulfonate esters | Alcohols | ethers |
| sulfonyl halides | amines/anilines | sulfonamides |
| sulfonyl halides | phenols/alcohols | sulfonate esters |
| *Activated esters, as understood in the art, generally have the formula —COΩ, where Ω is a good leaving group (such as oxysuccinimidyl (—OC₄H₄O₂) oxysulfosuccinimidyl (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations | | |
| # thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl). | | |

**Acyl azides can also rearrange to isocyanates

Methods to prepare reactive groups are well known in the art and their application to or modification for a particular purpose is within the ability of one of skill in the art (see, for example, Sandler and Karo, eds., Organic Functional Group Preparations, Academic Press, San Diego, 1989). Methods for preparing dye-conjugates are well documented in Haugland, Molecular Probes, Inc. Handbook of Fluorescent Probes and Research Chemicals, (9^{th} ed., September 2002) and Brinkley, Bioconjugate Chem., 3: 2 (1992). Conjugates typically result from mixing appropriate reactive compounds and the component to be conjugated in a suitable solvent in which both are soluble, using methods well known in the art, followed by separation of the conjugate from any unreacted components and by-products. The dye and specific binding moiety are typically combined under conditions of concentration, stoichiometry, pH, temperature and other factors that affect chemical reactions that are determined by both the reactive groups and the functional groups with which they react. These factors are generally well known in the art of forming dye-conjugates [Haugland et al., “Coupling of Antibodies with Biotin”, The Protein Protocols Handbook, J. M. Walker, ed., Humana Press, (1996); Haugland “Coupling of Monoclonal Antibodies with Fluorophores”, Methods in Molecular Biology, Vol. 45: Monoclonal Antibody Protocols, W. C. Davis, ed. (1995)]. For those reactive compounds that are photoactivated, conjugation includes illumination of the reaction mixture to activate the reactive compound. The labeled component is used in solution or lyophilized and stored for later use.

In an exemplary embodiment, preparation of dye-conjugates including peptide or proteins comprises first dissolving the protein to be conjugated in aqueous buffer at about 0.1-10 mg/mL at room temperature or below. Bicarbonate buffers (pH about 8.3) are especially suitable solvents for reactions of succinimidyl esters, phosphate buffers (pH about 7.2-8) for reactions of thiol-reactive functional groups and carbonate or borate buffers (pH about 9) for reactions of isothiocyanates and dichlorotriazines. The appropriate reactive dye is then dissolved in a nonhydroxylic solvent (usually DMSO or DMF) in an amount sufficient to give a suitable degree of conjugation when added to a solution of the protein to be conjugated.

The appropriate amount of dye for any protein or other component may be conveniently predetermined by experimentation in which variable amounts of the dye are added to the protein, the conjugate is chromatographically purified to separate unconjugated dye, and the dye-protein conjugate is tested in its desired application. Following addition of the reactive dye to the protein solution, the mixture is incubated for a suitable period (typically about 1 hour at room temperature to several hours on ice), the excess dye is removed by gel filtration, dialysis, HPLC, adsorption on an ion exchange or hydrophobic polymer or other suitable means. The dye-conjugate is then used in solution or lyophilized. In this way, suitable conjugates can be prepared from antibodies, antibody fragments, avidins, lectins, enzymes, proteins A and G, cellular proteins, albumins, histones, growth factors, hormones, and other proteins.

The approximate degree of dye substitution may be determined, for example, spectrophotometrically. The degree of dye substitution can be determined from the long wavelength absorption of the dye-protein conjugate by using the extinction coefficient of the un-reacted dye at its long wavelength absorption peak, the unmodified protein's absorption peak in the ultraviolet, and by correcting the UV absorption of the conjugate for absorption by the dye in the UV.

A dye-conjugate also can include a linker, L, between a dye moiety and a specific binding moiety. When present, the linker is a single covalent bond or a series of stable bonds. Thus, the reactive functional moiety may be directly attached (where the linker is a single bond) to a compound or attached through a series of stable bonds. When the linker is a series of stable covalent bonds the linker typically incorporates 1-20 non-hydrogen atoms selected, for example, from the group consisting of C, N, O, S, and P. In addition, the covalent linkage can incorporate a platinum atom, such as described in U.S. Pat. No. 5,714,327. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In an exemplary embodiment, the linker incorporates less than 15 nonhydrogen atoms and is composed of a combination of ether, thioether, thiourea, amine, ester, carboxamide, sulfonamide, hydrazide bonds and aromatic or heteroaromatic bonds. Typically the linker is a single covalent bond or a combination of single carbon-carbon bonds and carboxamide, sulfonamide or thioether bonds. The following moieties can be found in the linker: ether, thioether, carboxamide, thiourea, sulfonamide, urea, urethane, hydrazine, alkyl, aryl, heteroaryl, alkoxy, cycloalkyl and amine moieties. Examples of L include substituted or unsubstituted polymethylene, arylene, alkylarylene, arylenealkyl, or arylthio moieties.

Methods

Also disclosed is a staining method that provides reduced non-specific background staining of cells and tissues by dye-conjugates. The method includes contacting a cell or tissue with a dye-conjugate that directly or indirectly, specifically binds to a particular component of the cell or tissue. The method further includes contacting the cell or tissue with a blocking solution that includes a polymeric material other than a polymeric material included in the dye-conjugate and a solvent, such as water or a buffer. The cell or tissue can be contacted with the dye-conjugate and the polymeric material in any order. The polymeric material interferes with non-specific localization or binding of the dye-conjugate to cell and tissue components other than the particular component to which the dye-conjugate specifically binds. By interfering with non-specific localization or binding of the dye-conjugate, the polymeric material serves to reduce background staining by the dye-conjugate.

In a particular embodiment, a method for staining a cell or tissue with a dye-conjugate with reduced non-specific background staining by the dye-conjugate, comprising:
a) contacting the cell or tissue with the dye-conjugate to form a contacted sample, wherein the dye-conjugate specifically binds to a particular component of the cell or tissue;
b) contacting the contacted sample with a blocking solution to form a blocked sample, wherein the blocking solution comprises a polymeric material comprising multiple carboxylate, sulfate, sulfonate, phosphate, or phosphonate groups, wherein, if the polymeric material is a poly(amino acid), the polymeric material comprises a sulfated, sulfonated or phosphonated poly(amino acid), poly(aspartic acid) or poly(glutamic acid); and a buffer;
c) incubating the blocked sample for a sufficient amount of time for the dye-conjugate to specifically bind to a particular component of the cell or tissue and for the blocking solution to reduce non-specific background staining to form an incubated sample;
d) illuminating the incubated sample with an appropriate wavelength to form an illuminated sample; and,
e) observing the illuminated sample whereby the blocking solution reduces non-specific binding of the dye-conjugate to cell components other than the particular component to which the dye-conjugate specifically binds.

As mentioned above, the dye-conjugate may directly or indirectly specifically bind with a particular targeted component of a cell or tissue. Where the dye-conjugate binds directly and specifically with a particular targeted component, the dye-conjugate can include a primary antibody that specifically binds the particular component. Where the dye-conjugate binds indirectly and specifically with a particular component, the dye-conjugate can include a secondary antibody that specifically binds to a primary antibody that in turn specifically binds to the particular targeted component.

In some embodiments, a method is provided for staining a cell or tissue with a dye-conjugate so that there is reduced non-specific background staining by the dye-conjugate. The method includes contacting a cell or tissue with a blocking solution and then with the dye-conjugate, wherein the dye-conjugate specifically binds to a particular component of the cell or tissue. The polymeric material of the blocking solution comprises multiple carboxylate, sulfate, sulfonate, phosphate, or phosphonate groups, and if the polymeric material is a poly(amino acid), the poly(amino acid) comprises a sulfated, sulfonated or phosphonated poly(amino acid). Contacting the cell or tissue with the polymeric material reduces non-specific binding of the dye-conjugate to cell components other than the particular component to which the dye-conjugate specifically binds. In other embodiments, a method is provided for staining a cell or tissue with a dye-conjugate, wherein any resulting background staining of cell or tissue components can be reduced or eliminated by contacting the cell or tissue with a polymeric material or materials after the staining protocol has been completed.

Any of the polymeric materials disclosed above may be utilized in the disclosed methods. In a particular embodiment of the method, the polymeric material is dissolved in a solution and has a concentration from about 0.1 mg/mL to about 20 mg/mL, for example, a concentration from about 0.5 mg/mL to about 10 mg/mL, such as a concentration from about 1 mg/mL to about 5 mg/mL. In more particular embodiments the solution in which the polymeric material is dissolved includes a buffer that does not substantially interfere with a specific binding reaction of a dye-conjugate. Suitable buffers are discussed above. The solution may further include one or more of a detergent or a preservative. In a particular embodiment the blocking solution comprises a polymeric material, PBS pH 7.2 and 2 mM sodium azide as the preservative.

The dye-conjugate used in the disclosed methods may be any dye-conjugate, in particular the dye-conjugates disclosed above or in the examples that follow. In a particular embodiment the dye conjugate comprises, but it not limited to, an avidin or binding fragment thereof; a streptavidin or binding fragment thereof; an antibody or a binding fragment thereof; a receptor protein or a binding fragment thereof; a protein A or a binding fragment thereof; a protein G or a binding fragment thereof; a nucleic acid; an enzyme or binding fragment thereof; a metal ion chelator or a metal-chelating fragment thereof; or, a lectin or a binding fragment thereof. In another embodiment, the dye conjugate comprises a fluorescent dye. In one aspect the fluorescent dye is anionic or neutral and include, but are not limited to, a sulfonated xanthene dye, sulfonated cyanine dye, sulfonated pyrene dye or sulfonated coumarin dye. Examples of such anionic or neutral dyes include fluorescein, tetramethylrhodamine, Allophycocyanin, R-phycoerythrin and dyes sold under the trade names Oregon Green®, Cascade Blue®, Alexa Fluor®, Texas Red®, Cascade Yellow™, Marina Blue®, DyeMer™, Atto, Cy® and Dy dyes.

In particular embodiments, the background staining that is reduced by the method includes non-specific staining of cell nuclei or non-specific staining of white matter in neuronal tissue. In other particular embodiments, the polymeric material reduces a level of non-specific staining of the cell nuclei in the absence of the composition to a lower level of non-specific staining, for example, a level of non-specific staining of the cell nuclei that is at least 20% lower (such as 50%, 75% or 90% lower) than the level of non-specific staining in the absence of the composition. In more particular embodiments, the levels of non-specific staining are levels of fluorescence emitted from at least a portion of a sample, such as a portion including the surface of a nucleus or including white matter tissue. For example, the average relative pixel intensity over one or more regions (such as regions of 10 or more, 20 or more, 50 or more, 100 or more, or 1000 more pixels) exhibiting background staining may be reduced by at least 20%, at least 50%, at least 75%, 90% or 95%. In other particular embodiments, specific binding of a dye-conjugate is not substantially affected by treatment according to the disclosed methods. For example, the specific binding of the dye-conjugate may be reduced by less than 5%, for example, less than 1%, such as less than 0.1%.

In the disclosed methods, the polymeric material(s) also can be used with or without an additional step, which is typically employed to reduce background staining after fixation of cells or tissues. In fact, the use of the disclosed polymeric materials can act as a substitute for a BSA or other blocking materials commonly used. The polymeric material(s) also can be mixed with either a primary or secondary antibody. For example, a cell or tissue can be contacted simultaneously with the polymeric material and the dye-conjugate, which may be either a primary or secondary antibody.

The sample includes, without limitation, any biological derived material that is thought to contain a target ligand. Typically the sample is biological in origin and comprises tissue, cell or a population of cells, cell extracts, cell homogenates, purified or reconstituted proteins, recombinant proteins, bodily and other biological fluids, viruses or viral particles, prions, subcellular components, or synthesized proteins. The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells such as bacteria, yeast, fungi, mycobacteria and mycoplasma, and eukaryotic cells such as nucleated plant and animal cells that include primary cultures and immortalized cell lines. Typically prokaryotic cells include E. coli and S. aureus. Eukaryotic cells include without limitation ovary cells, epithelial cells, circulating immune cells, β cells, hepatocytes, and neurons.

Samples that are treated with disclosed polymeric materials to reduce background staining can be fixed (dead) cells and tissues. Alternatively, staining of live cells may benefit from the disclosed methods and compositions. Regardless, cell samples and tissue sections are commonly probed for the presence of antigens and other molecular targets with specific binding agents such as dye-conjugates. Such dye-conjugates incorporate moiety that enables subsequent target (ligand) visualization. This target visualization or localization is commonly referred to as cell and tissue staining and it can be realized by numerous immunocytochemical and histochemical techniques familiar to those of ordinary skill in the art. Fixation of the cell or tissue sample improves localization of particularly targeted structures and molecules.

Although other known methods of cell and tissue preparation, fixation and permeabilization exist, the following are general methods for preparing, fixing, and permeabilizing cell and tissue samples prior to cytochemical and histochemical target localization procedures.

Both cells and tissues isolated from an in vivo source and those obtained from in vitro cultures may be stained with reduced background by the disclosed methods. In some embodiments, cells cultured in vitro or thin sections of tissue cut from tissue samples of interest with a microtome are used. Cells for staining can be primary cultures or cell lines, derived directly from various tissues, and may be from epithelial, endothelial, neuronal, lymphoid, muscle, hepatic or many other tissues. Cell lines derived from tumors and other long-lived or immortal cells can also be grown in culture for prolonged periods and used repeatedly for experimental analyses. Cells capable of growing while attached to a surface are typically propagated on sterile glass coverslips or a variety of other substrates. Many types of cells can also be grown in suspension culture and transferred to an appropriately prepared semi- or solid surface prior to processing. Cells are typically grown in a variety of semi-defined or defined culture media well known in the art. Tissue samples for staining can be obtained from healthy and/or diseased animals following appropriate anesthesia and euthanasia procedures, by various biopsy techniques, at autopsy, or by withdrawal of blood or bone marrow from a living subject. However, regardless of the sample source, a majority of cell and tissue staining procedures include both fixation and permeabilization of the sample before staining.

Fixation of the cells or tissues helps improve the accuracy of the determination of the spatial distribution of the target substance(s) in the sample. The goal of most fixation techniques is to minimize change to the cell or tissue structure as well as their chemical characteristics. For example, improper or inadequate fixation can lead to the migration of target molecules or antigens away from their normal cellular locations and their deposition in inappropriate sites. Fixation techniques also typically strive to maintain, or even promote, the antigenicity of the target molecules.

Many fixatives and mixtures thereof are known. Fixatives containing cross-linking agents like formaldehyde, paraformaldehyde, glutaraldehyde, carbodiimides, N-hydroxysuccinimidyl esters, picric acid, and trinitroresocinol are very commonly used. Solvents often used for fixation are methanol, ethanol, and acetone. One or a mixture of these agents in solution is usually applied directly to the cells before further processing. In the case of tissues, living, anesthetized source animals are often perfused transcardially with buffered solutions of fixatives prior to harvesting of the samples. Tissue samples can also be immersed in fixative solutions for various lengths of time. Since no single fixation technique is optimal for all cells or tissues, those of ordinary skill in the art typically develop their own fixation protocols to optimize their chances of successfully visualizing the desired cell or tissue targets with staining techniques.

After fixation and before staining, cell and tissue samples are usually treated with agents that increase the permeability of membrane-bound cellular structures. This is desirable because most fixatives actually decrease membrane permeability and may make many target molecules less accessible to antibodies and other probes. Also, most cytochemical and histochemical probes such as antibodies are too large to freely diffuse into fixed cellular structures without first enhancing their permeability (permeabilization). Common permeabilization agents are nonionic detergents such as Triton X-100, Brij 35, Nonidet P-40, and Tween-20 and other Tween derivatives. Such detergents are popular because they usually do not cause protein denaturation. The solvents listed above also can be used as permeabilization agents because of their ability to extract membrane lipids that act as diffusion barriers to applied aqueous probes. Detergent-like substances, for example, lysolecithins and saponins also can be used for permeabilization because they disrupt the organization of membrane lipids such that cell permeability is enhanced. As with fixation, those of ordinary skill in the art typically develop application-specific permeabilization protocols that meet their specific needs.

After fixation and permeabilization, if employed, the sample can then be treated using a staining protocol. Most staining protocols begin with a step where the fixed and permeabilized sample(s) is pre-incubated with a solution containing constituents that bind to and block cellular sites likely to bind the antibody or other probe nonspecifically. This step is commonly referred to as “blocking”. Such nonspecific binding, which results in nonspecific or background staining, is commonly encountered and is often difficult or impossible to completely eliminate. Typical blocking solutions consist of high concentrations of heterogeneous, often proteinaceous components or mixtures thereof such as serum albumins, unfractionated blood sera, gelatin, milk proteins, detergents and the like. The ability of such blocking solutions to mitigate background staining is highly application-specific and often suboptimal. The disclosed compositions and methods for blocking background staining described herein provide a solution to many such background staining problems.

For those samples that are mounted on microscope slides, a mounting medium is typically applied after the staining protocol and prior to the placement of a coverslip and subsequent illumination with an apprioriate wavelength. Many mounting mediums are known to one of skill in the art and include water and buffers. Due to the photolabile properties of many dye moieties, mounting mediums are often employed that reduce the photobleaching of the dye moieties due to prolonged excitation. Such mounting mediums include SlowFade and ProLong (Invitrogen Corp.). See, Examples 9 and 10.

However, at any time after staining of the sample with the dye-conjugate, the sample can be illuminated with a wavelength of light that is selected to give a detectable optical response (a signal) from the dye moiety. The detectable optical response can be any optical response, for example, aborption or emission of light by the dye moiety, or a change in a fluorescence lifetime of the dye moiety.

Equipment for illuminating a sample includes, but is not limited to, hand-held ultraviolet lamps, mercury arc lamps, xenon lamps, lasers and laser diodes. Such illumination sources are often optically integrated into laser scanners, fluorescent microplate readers, fluorescence-activated cell sorters, fluorescence microscopes and standard or microfluorometers.

The detectable optical response may be detected by any means for detecting an optical response, including visual inspection and electronic detection. Detection of the response may be accomplished, for example, by using any of the following devices: CCD camera, video camera, photographic film, laser-scanning devices, fluorometers, photodiodes, quantum counters, epifluorescence microscopes, scanning microscopes, flow cytometers, fluorescence microplate readers, or by a means for amplifying the signal such as photomultiplier tubes. Where the sample is examined using a flow cytometer, examination of the sample optionally includes sorting portions of the sample according to their fluorescence response. The degree and/or location of signal, compared with a standard or expected response, indicates whether and to what degree the sample possesses a given characteristic, for example, a desired target.

When an indirectly detectable label is used, the step of illuminating typically further includes addition of a reagent that facilitates a detectable optical signal, for example, a colorogenic, luminogenic or fluorogenic enzyme substrate.

C. Kits

Kits including a disclosed polymeric material and instructions for using the polymeric material to reduce non-specific background staining also are provided. The instructions are typically written instructions that may be printed instructions (such as on the packaging, or as a separate insert contained in the kit) or instructions stored on a computer readable medium such as a CD or a diskette. Such written instructions may include one or more diagrams or pictures illustrating steps of the methods they describe or typical results obtained with the polymeric material of the kit.

In some embodiments, a kit is provided for reducing non-specific staining of a cell or tissue by a dye-conjugate. The kit includes a blocking solution comprising a polymeric material bearing multiple carboxylate, sulfate, sulfonate, phosphate, or phosphonate groups and a buffer. However, if the polymeric material is a poly(amino acid), the polymeric material comprises a sulfated, sulfonated or phosphonated poly(amino acid). The kit also includes instructions explaining how to use the polymeric material to reduce non-specific staining of the cell or tissue by the dye-conjugate.

The polymeric material included in the kit may be dissolved in a solution at a concentration sufficient to provide a detectable reduction in non-specific background staining by a dye-conjugate, such as at a concentration from about 0.1 mg/mL to about 20 mg/mL, for example, a concentration from about 0.5 mg/mL to about 10 mg/mL, such as a concentration from about 1 mg/mL to about 5 mg/mL. The solution can be a buffer that does not substantially interfere with a specific binding reaction of a dye-conjugate. Particular examples of suitable buffers were provided above and in the examples that follow. The solution may further include a detergent or a preservative, such as the detergents and preservatives discussed above or in the following examples.

In other embodiments, the kit further includes a dye-conjugate, which may be any dye-conjugate, and in particular, any of the dye-conjugates disclosed above or in the examples that follow. Particular dye-conjugates may include an avidin or binding fragment thereof; a streptavidin or binding fragment thereof; an antibody or a binding fragment thereof; a receptor protein or a binding fragment thereof; a protein A or a binding fragment thereof; a protein G or a binding fragment thereof; a nucleic acid; an enzyme or binding fragment thereof; a metal ion chelator or a metal-chelating fragment thereof; or, a lectin or a binding fragment thereof. In particular embodiments, the dye-conjugate comprises a fluorescent dye, for example, neutral fluorescent dye, such as a fluorinated dye, or an anionic fluorescent dye, such as a sulfonated fluorescent dye.

In some embodiments of the kit, the polymeric material comprises a synthetic polymer, a carbohydrate polymer or a sulfated poly(amino acid). In particular embodiments, the polymeric material included in the kit includes a polystryrene or copolymer thereof; a polyacrylamide or copolymer thereof; a polyvinylene or copolymer thereof; a polyalkalene or copolymer thereof; a polyaniline or copolymer thereof; a polyphenylalkylene of copolymer thereof; a heparin or derivative thereof; a dextran or derivative thereof; a suramin or derivative thereof; carrageenan or derivative thereof; a cyclodextrin other than sulfobutylether beta-cyclodextrin or derivative thereof; a cellulose or derivative thereof; a pentosan or derivative thereof; a dextrin or derivative thereof; a laminarin or derivative thereof; a dermatan or derivative thereof; a chitin or derivative thereof; a chitosan or derivative thereof; a curdlan or derivative thereof; a pullulan or derivative thereof; a keratan or derivative thereof; a fucoidan or derivative thereof; a ficoll or derivative thereof, a xylan or derivative thereof; an amylose or derivative thereof; a galactan or derivative thereof; a mucin or derivative thereof; a galactomannan or derivative thereof; a mannan or derivative thereof; a glucan or derivative thereof; a fucan or derivative thereof; a heparaosan or derivative thereof; a rhamnan or derivative thereof; a catechin or derivative thereof; or a calixarene or derivative thereof. More particular examples of polymeric materials are provided above and in the examples that follow.

In a particular embodiment, the polymeric material of the kit includes poly(sodium 4-styrenesulfonic acid), poly(4-styrenesulfonic acid-co-maleic acid), poly(2-acrylamido-2-methyl-1-propanesulfonic acid), poly(vinylsulfate), poly(vinylsulfonic acid), poly(vinylphosphate), poly(vinylphosphonic acid), poly(anilinesulfonic acid), poly(anetholesulfonic acid), heparin, heparin-like substance, deaminated heparin, chondroitin sulfate, dextran sulfate, sulfopropyl-beta-cyclodextrin, or beta-cyclodextrin tetradecasulfate. In more particular embodiments, the polymeric material of the kit includes heparin or dextran sulfate, and in even more particular embodiments, the polymeric material is a combination of heparin and dextran sulfate. The heparin is desirably porcine Type 1-A heparin and the dextran sulfate is desirably a dextran sulfate having an average molecular weight of from about 5,000 to about 1,000,000, for example, an average molecular weight of about 15,000 to about 100,000.

In a very particular embodiment, the kit includes a solution comprising, heparin at a concentration of 0.1-20 mg/mL and dextran sulfate at a concentration of 0.1-20 mg/mL dissolved in a buffer and also containing a preservative.

In an alternative embodiment, the kit further includes a dye-conjugate. Thus, in one embodiment, a kit is provided for specific staining of a component of a cell or tissue with reduced nuclear background staining, wherein the kit includes a dye-conjugate and a polymeric material such as those disclosed above and below in the examples. In a particular embodiment, the kit includes a dye-conjugate that includes a sulfonated fluorescent dye moiety and a polymeric material bearing multiple sulfate or sulfonate groups. The kit can further include one or more of written instructions, an anti-fade reagent, a mounting medium, glass slides, cover slips, chambered culture wells, a buffer (which can be provided separately or as part of a solution containing either or both of the dye-conjugate and the polymeric material), a preservative, and a detergent.

A detailed description of the invention having been provided above, the following examples are given for the purpose of illustrating the invention and shall not be construed as being a limitation on the scope of the invention or claims.

### EXAMPLES

### Example 1

### Preparation of Blocking Solutions

For determination of dye background reducer (DBR) activity, polymeric materials were dissolved in phosphate-buffered saline (10 mM potassium phosphate/150 mM NaCl, pH 7.2, containing 2 mM sodium azide) (PBS). Typical solutions included polymeric material(s) dissolved at a final concentration of 1 mg/ml, although concentrations greater or less than 1 mg/mL of one or multiple components can also be used. For example, one of the DBR solutions contained heparin and dextran sulfate at final concentrations of 2 mg/mL and 6 mg/mL, respectively. After the compounds were fully dissolved, the pH of the solutions was determined. In some instances, if the pH of the resulting solution was between 6.5 and 7.2, an aliquot was withdrawn and adjusted to pH 1.5-3.0 by adding 6M HCl. In other instances, if the pH of the resulting solution was between 1.5 and 3.0, an aliquot was withdrawn and adjusted to pH 6.5-7.2 by adding 6M NaOH. In either or both of these pH ranges, compounds were then tested for their ability to inhibit non-specific background staining by fluorescent dye-conjugates.

### Example 2

### Inhibition of Dye Background Staining in Fixed and Permeabilized Cultured Cells by DBR Solutions Used Before Staining with Streptavidin-Fluorescent Dye-Conjugates

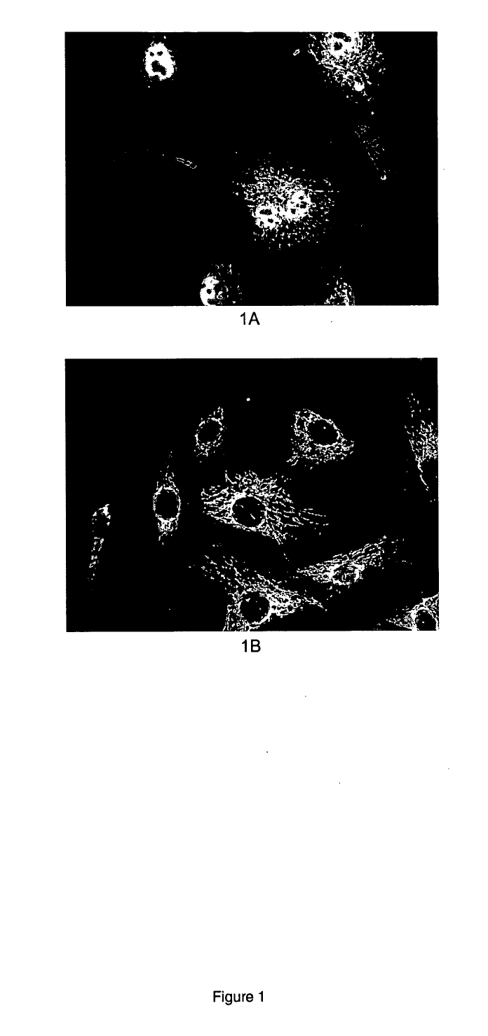

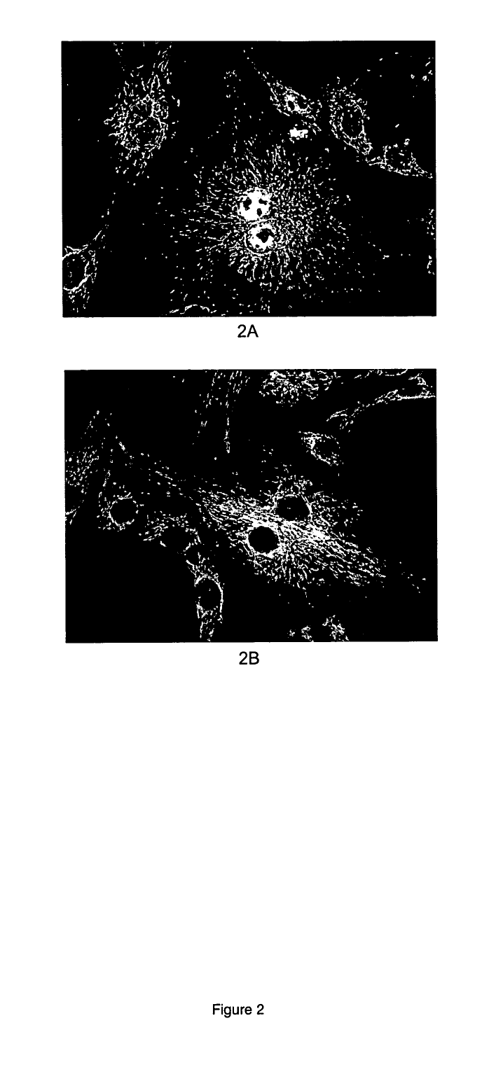

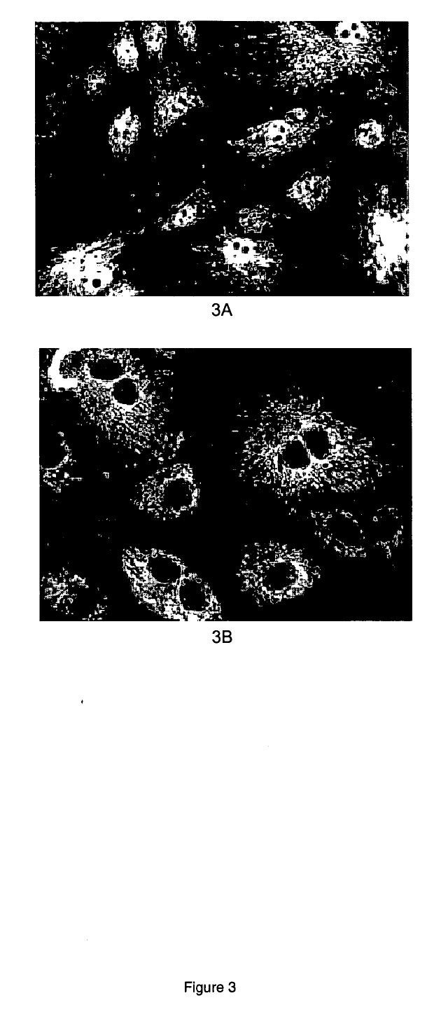

Bovine pulmonary artery endothelial cells or HeLa cells were grown in Dulbecco's modified minimal essential Eagle's medium supplemented with 20% (v/v) fetal bovine serum, plated onto 18 mm² glass coverslips in 100 mm plastic Petri dishes, and cultured to 50-60% confluency. These cultures were fixed in 3.7% (w/v) formaldehyde (or in methanol or ethanol) in PBS at 23-25° C. (room temperature (RT)) for 5 min. After rinsing 3 times with PBS, the cells were then permeabilized with 0.2% (v/v) Triton X-100 in PBS for 5 min at RT. The cells were then washed 3 times with PBS and incubated for 30 min at RT with 1% (w/v) bovine serum albumin (BSA) in PBS. After 3 more rinses with PBS, the cells were incubated for 30 min at RT with either a DBR (200 μl per coverslip), e.g. PBS containing heparin, dextran sulfate, poly(vinylsulfate) or poly(styrenesulfonic acid) at 1 mg/ml prepared as described in Example 1, or with equivalent volumes of PBS as the control. The cells were washed 3 more times with PBS and stained with a streptavidin-fluorescent dye-conjugate at a final concentration of 10 μg/ml for 30 min at RT. After washing 3 times with PBS, the coverslips were mounted on glass microscope slides with Cytoseal™-60 mounting medium. Fluorescent staining was observed with a Nikon Eclipse E400 fluorescence microscope equipped with filters appropriate for the dye(s) used. Images were acquired with a MicroMAX digital camera incorporating an X1300 1030 charged-coupled device controlled by MetaMorph imaging software. Cells not pre-treated with a DBR typically showed strong or weak nonspecific fluorescent staining of their nuclei in addition to the expected bright specific fluorescent staining of biotinylated proteins in their mitochondria. Cells treated with a maximally effective DBR typically showed only the specific mitochondrial staining. Exemplary images of these tests are shown in FIGS. 1-3. The reduction of background staining by the polymeric material is dramatically demonstrated by comparing FIG. 1A with FIG. 1B, by comparing FIG. 2A with FIG. 2B, and by comparing FIG. 3A and FIG. 3B.

In summary, thirty-seven out of the 46 streptavidin-dye-conjugates tested produced either strong (27/37) or weak (10/37) nuclear background staining which was blocked by pre-treatment with the DBR heparin at 1 mg/ml (Table 3). Cell staining with 9/46 of the fluorescent dye-streptavidin conjugates did not result in nuclear background staining. In these cases, pre-treating the cells with a DBR had no effect on specific fluorescent mitochondrial staining (Table 4).

The relative potency of each of the tested compounds at reducing nuclear background staining was scored visually using the following scale: +++=maximal blocking (see FIGS. 1B, 2B and 3B for examples of maximal blocking), ++=moderate blocking, +=weak blocking, and — inactive (see Table 5). Twenty-four of the 42 compounds tested were evaluated at pH 6.5-7.2 and at pH 1.5-3.0. Seven out of these 24 compounds showed an increase in relative blocking potency when their pH was lowered to pH 1.5-3.0 from pH 6.5-7.2; compounds 9-11 changed from [symbol] to +++, compound 33 changed from ++ to +++, compounds 35 and 37 changed from — to +, and compound 39 changed from — to ++ (Table 5). The pH of the solutions had no effect on the relative background blocking ability of the remaining 17 compounds in the group. A complete summary of these data is shown in Table 5. Those sulfonated, sulfated, phosphorylated, and phosphonated compounds indicated to exhibit no visually detectable reduction in background may nonetheless exhibit a reduction detectable by other means, such as electronically. Furthermore, such compounds exhibiting no visually detectable reduction in background at the pHs tested may nonetheless exhibit a reduction in background at other pHs.

TABLE 3
Fluorescent Dyes Causing Background Staining¹

| | Dye Type | Background Reduced² |
|---|---|---|
| Strong Background | | |
| Dye 1 | Anionic | yes |
| | xanthene | |
| Dye 2 | Anionic | yes |
| | xanthene | |
| Dye 3 | Anionic | yes |
| | xanthene | |
| Dye 4 | Neutral | yes |
| | xanthene | |
| Dye 5 | Neutral | yes |
| | xanthene | |
| Dye 6 | Neutral | yes |
| | oxazol | |
| Dye 7 - Dy 565 | proprietary | yes |
| Dye 8 - Dy 630 | Neutral | yes |
| Dye 9 - Atto 590 | proprietary | yes |
| Dye 10 - Atto 610 | proprietary | yes |
| Dye 11 | Anionic | yes |
| | pyrene | |
| Dye 12 | Anionic | yes |
| | pyrene | |
| Dye 13 | Anionic | yes |
| Dye 14 | Anionic | yes |
| | xanthene | |
| Dye 15 | Anionic | yes |
| | xanthene | |
| Dye 16 | Anionic | yes |
| | xanthene | |
| Dye 17 | Anionic | yes |
| | cyanine | |
| Dye 18 | Anionic | yes |
| | xanthene | |
| Dye 19 | Anionic | yes |
| | xanthene | |
| Dye 20 | Anionic | yes |
| | xanthene | |
| Dye 21 | Anionic | yes |
| | xanthene | |
| Dye 22 | Anionic | yes |
| | xanthene | |
| Dye 23 | Anionic | yes |
| | cyanine | |
| Dye 24 | Anionic | yes |
| | cyanine | |
| Dye 25 | Anionic | yes |
| | cyanine | |
| Dye 26 | Anionic | yes |
| | cyanine | |
| Dye 27 | Anionic | yes |
| | cyanine | |
| Weak Background | | |
| Dye 28 - Cy ™ 5 | Anionic | yes |
| | Cyanine | |
| | dye | |
| Dye 29 - Dy 635 | Neutral | yes |
| Dye 30 | Neutral | yes |
| | coumarin | |
| Dye 31 | Neutral | yes |
| | xanthene | |
| Dye 32 | Anionic | yes |
| | xanthene | |
| Dye 33 allophycocyanin | Neutral | yes |
| | protein | |
| Dye 34 R-phycoerythrin | Neutral | yes |
| | protein | |
| Dye 35 | Anionic | yes |
| | xanthene | |
| Dye 36 | Anionic | yes |
| | xanthene | |
| Dye 37 | Anionic | yes |
| | xanthene | |

¹all dyes were conjugated to streptavidin and tested at 10 μg/ml
²tested with heparin at 1 mg/ml as the DBR

TABLE 4
Background-Free Fluorescent Dyes

| Dye¹ | Dye Type | Staining Change² |
|---|---|---|
| Dye 38 | Neutral | no |
| | coumarin | |
| Dye 39 | Neutral | no |
| Dye 40 - Cy ™ 3 | Anionic | no |
| | cyanine | |
| | dye | |
| Dye 41 | Neutral | no |
| | coumarin | |
| Dye 42 | Neutral | no |
| | xanthene | |
| Dye 43 | Neutral | no |
| | xanthene | |
| Dye 44 | Neutral | no |
| | xanthene | |
| Dye 45 - Dy 550 | Proprietary | no |
| Dye 46 - Dy 610 | Proprietary | no |

¹all dyes were conjugated to streptavidin and tested at 10 μg/ml
²tested with heparin at 1 mg/ml as the DBR

TABLE 5
Polymeric Materials Reducing Dye Background Staining

| | | Reduces | |
|---|---|---|---|
| | | Background | |
| Compounds Tested¹ | CAS # | pH 6.5-7.2 | pH 1.5-3.0 |

Polystyrene-based compounds

| | | | |
|---|---|---|---|
| 1. poly(sodium 4-styrenesulfonic acid) MW = 70,000 | 25704- | +++² | +++ |
| | 18-1 | | |
| 2. poly(sodium 4-styrenesulfonic acid) MW = 200,000 | 25704- | +++ | +++ |
| | 18-1 | | |
| 3. poly(sodium 4-styrenesulfonic acid) MW = 1,000,000 | 25704- | ++ | ++ |
| | 18-1 | | |
| 4. poly(lithium 4-styrenesulfonic acid) MW = 75,000 | 9016-91-5 | +++ | +++ |
| 5. poly(4-styrenesulfonic acid-co-maleic acid) MW = 20,000 | 68037- | +++ | +++ |
| | 40-1 | | |

Polyacrylamide-based compounds

| | | | |
|---|---|---|---|
| 6. poly(2-acrylamido-2-methyl-1-propanesulfonic acid) MW = 2,000,000 | 27119- | +++ | +++ |
| | 07-9 | | |
| 7. poly(2-acrylamido-2-methyl-1-propanesulfonic acid-co-styrene) | 51121- | — | — |
| | 85-8 | | |

Polyvinyl-based compounds

| | | | |
|---|---|---|---|
| 8. poly(vinylsulfate) practical grade | 26182- | nt | ++ |
| | 60-5 | | |
| 9. poly(vinylsulfate) MW = 170,000 | 26182- | — | +++ |
| | 60-5 | | |
| 10. poly(vinylsulfonic acid) | 25053- | — | +++ |
| | 27-4 | | |
| 11. poly(vinylphosphate) | 29690- | — | +++ |
| | 74-2 | | |
| 12. poly(vinylphosphonic acid) | 27754- | +++ | +++ |
| | 99-0 | | |

Other synthetic polymer compounds

| | | | |
|---|---|---|---|
| 13. poly(anilinesulfonic acid) MW = 10,000 | 167860- | +++ | +++ |
| | 86-8 | | |
| 14. poly(anetholesulfonic acid) MW = 10,000 | 52993- | +++ | +++ |
| | 95-0 | | |
| 15. poly[di(ethyleneglycol)/cyclohexanedimethanol-alt-isophthalic acid, sulfonated] | | — | — |

Sulfated Carbohydrate Polymers

| | | | |
|---|---|---|---|
| 16. heparin (porcine, type I-A) | 9041-08-1 | +++ | +++ |
| 17. heparin (porcine, crude, unbleached) | 9041-08-1 | ++ | Nt |
| 18. heparin (porcine, low Ca⁺⁺) | 9041-08-1 | ++ | Nt |
| 19. heparin (porcine) MW = 3,376 | 9041-08-1 | ++ | Nt |
| 20. heparin (porcine) MW = 5,060 | 9041-08-1 | ++ | Nt |
| 21. heparin (bovine) | 9041-08-1 | ++ | Nt |
| 22. heparin-like substance (porcine, mesoglycan) | | + | Nt |
| 23. deaminated heparin (porcine) | | + | Nt |
| 24. chondroitin sulfate (bovine) | 9007-28-7 | ++ | Nt |

Non-sulfated and poorly sulfated compounds

| | | | |
|---|---|---|---|
| 25. hyaluronic acid (bovine) | 9004-61-9 | — | Nt |
| 26. de-N-sulfated heparin (porcine) | 61932- | — | Nt |
| | 66-9 | | |
| 27. heparan (bovine) | 57459- | — | — |
| | 72-0 | | |

Sulfated dextrans

| | | | |
|---|---|---|---|
| 28. dextran sulfate MW <15,000 | 9011-18-1 | ++ | Nt |
| 29. dextran sulfate MW = 40,000-50,000 | 9011-18-1 | +++ | +++ |
| 30. dextran sulfate MW = 500,000 | 9011-18-1 | ++ | Nt |

Cyclodextrin derivatives

| | | |
|---|---|---|
| 31. sulfobutylether beta-cyclodextrin | — | — |
| 32. sulfopropyl-beta-cyclodextrin | + | Nt |
| 33. beta-cyclodextrin tetradecasulfate | ++ | +++ |

Synthetic Neutral Polymers

| | | | |
|---|---|---|---|
| 34. poly(1-hexene-sulfone) MW = 4,609,908 | 34903- | — | — |
| | 07-6 | | |
| 35. poly(1-octene-sulfone) MW = 6,134,863 | 30795- | — | + |
| | 19-8 | | |
| 36. poly(1-dodecene-sulfone) MW = 14,000,000 | 33990- | — | — |
| | 99-7 | | |
| 37. poly(1-tetradecene-sulfone) MW = 21,002,678 | 33991- | — | + |
| | 00-3 | | |
| 38. poly(ethyleneglycol)-4-nonylphenyl-3-sulfopropyl ether | 119438- | — | Nt |
| | 10-7 | | |

Miscellaneous Compounds

| | | | |
|---|---|---|---|
| 39. suramin (low MW synthetic heparin analogue) | 129-46-4 | — | ++ |
| 40. protamine sulfate (sulfated cationic salmon protein) | 53597- | — | Nt |
| | 25-4 | | |
| 41. phytic acid (phosphorylated inositol ester) | 14306- | — | Nt |
| | 25-3 | | |
| 42. NaSO₄ (inorganic salt) | 7757-82-6 | — | Nt |

¹all compounds were tested at 1 mg/ml
²blocking potency: +++ = maximal, ++ = moderate, + = weak, — = inactive, nt = not tested

### Example 3

### Inhibition of Dye Background Staining in Fixed and Permeabilized Cultured Cells by Solutions Used after Staining with Streptavidin-Fluorescent Dye-Conjugates

Bovine pulmonary artery endothelial or HeLa cells were cultured on glass coverslips, fixed, permeabilized, and treated with a BSA solution as described in Example 2. The cells were washed 3 more times with PBS and then stained with a streptavidin-fluorescent dye-conjugate at a final concentration of 10 μg/ml for 30 min at RT. After 3 rinses with PBS, the cells were then incubated for 30 min at RT with either a DBR solution as described in Example 2, or with equivalent volumes of PBS as the control. After washing 3 times with PBS, the coverslips were mounted as described in Example 2. Fluorescence microscopy and imaging were conducted as described in Example 2. As described in Example 2, cells not treated with a DBR typically showed strong nonspecific fluorescent staining of their nuclei in addition to the expected specific fluorescent staining of biotinylated proteins in their mitochondria. Cells treated with a DBR after the staining process was completed typically showed only the specific mitochondrial staining as described in Example 2 and as shown in FIGS. 1-3. This example demonstrates that the disclosed polymeric materials can be applied to a sample after staining with a dye-conjugate, and still provide reduction in non-specific binding of the dye-conjugate.

### Example 4

### Inhibition of Dye Background Staining in Fixed and Permeabilized Cultured Cells by Solutions Used in Conjunction with Antibody-Fluorescent Dye-Conjugates

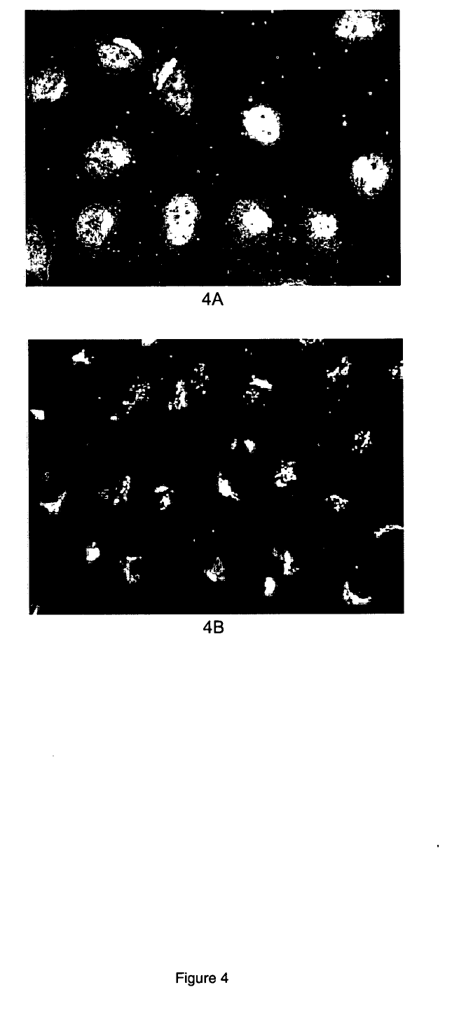

Bovine pulmonary artery endothelial or HeLa cells were cultured on glass coverslips, fixed, permeabilized, and treated with a BSA solution as described in Example 2. The cells were washed 3 more times with PBS and then incubated for 30 min at RT with a DBR. The cells were washed with PBS and then incubated for 1 hr at RT with a mouse monoclonal anti-golgin antibody (primary antibody) (0.5 μg/mL) that binds to the Golgi apparatus. After washing with PBS, the cells were incubated for 1 hr at RT with a fluorescent goat anti-mouse IgG (secondary antibody) (5 μg/mL), e.g. goat anti-mouse IgG-Alexa Fluor 488, that binds to the anti-golgin antibody. The cells were washed with PBS, mounted, and observed by fluorescence microscopy as described in Example 2. Control cells not treated with a DBR typically showed strong nuclear and cytoplasmic background staining in addition to the specific Golgi staining (FIG. 4A). Cells treated with a DBR before applying the antibodies typically showed only the specific Golgi staining (FIG. 4B). Although similar results were obtained when the DBR was mixed with either the primary or the secondary antibody, the DBR was most effective when it was applied separately as described above.

### Example 5

### Inhibition of Dye Background Staining of White Matter in Fixed and Permeabilized Brain Tissue Sections by DBR Solutions Used Before Staining

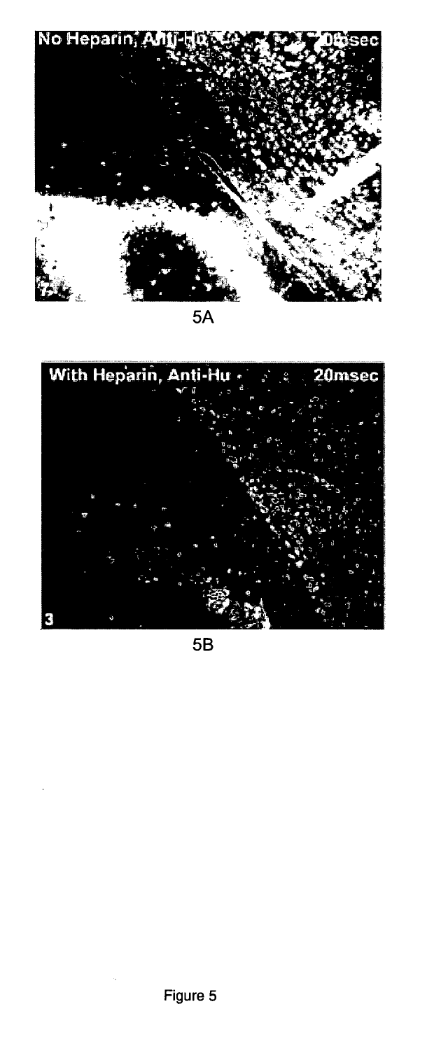

Perfused and frozen mouse brain tissue was transferred to Peel-Away molds, embedded in Sakura Finetek's Tissue-Tek OCT compound, and frozen in liquid nitrogen. Coronal sections (10 μm) of the hippocampus were cut with a Leica CM3050S cryostat, collected on Superfrost Plus glass microscope slides, air-dried, desiccated, and stored at −85° C. For staining, sections were brought to RT and rehydrated for 15 min in PBS. The tissue sections were permeabilized for 15 min at RT with PBS containing 0.2% (w/v) BSA and 0.2% (v/v) Triton X-100 (PBT) and then incubated for 30 min at RT with 5% (v/v) normal goat serum in PBT. After washing with PBS, the sections were incubated for 30 min at RT with either a DBR as described in Example 2 or with PBS as a control. After washing again with PBS, the sections were then incubated overnight at 4° C. with, e.g. a monoclonal mouse anti-Hu C/D antibody at 10 μg/mL, diluted in PBT. The slides were then washed 4 times for 15 min each with PBT and then stained for 2 hr at RT with a fluorescein conjugate of goat anti-mouse IgG at 5 μg/mL, diluted in PBT. The slides were then washed a final time in PBS, mounted as described in Example 2, and evaluated by fluorescence microscopy as described in Example 2. Tissue sections not treated with a DBR typically showed strong nonspecific fluorescent staining of white matter regions in addition to specific fluorescent staining of the Hu C/D target in the cell bodies. White matter background staining was typically reduced to autofluorescence levels in sections treated before staining with a DBR (FIG. 5). There was no significant change in the resolution and fluorescence intensity of the specific cell body staining in brain sections pre-treated with a DBR.

### Example 6

### Inhibition of Dye Background Staining of White Matter in Fixed and Permeabilized Brain Tissue Sections by a DBR Solution after Staining

Tissue sections of fixed and permeabilized mouse hippocampus were prepared for staining as described in Example 5. After washing with PBS, the sections were incubated overnight at 4° C. with, e.g. a monoclonal mouse anti-Hu C/D antibody at 10 μg/mL, diluted in PBT. The slides were then washed 4 times for 15 min each with PBT and stained for 2 hr at RT with a fluorescent dye-conjugate of goat anti-mouse IgG at 5 μg/mL, diluted in PBT. The sections were then washed thoroughly with PBS and incubated for 30 min at RT with either a DBR as described in Example 5 or with PBS as a control. The sections were washed a final time in PBS, mounted as described in Example 2, and fluorescence microscopy/imaging was conducted as described in Example 2. Tissue sections not treated with a DBR after staining typically showed strong nonspecific fluorescent staining of white matter regions in addition to specific fluorescent staining of histones in their nuclei. White matter background staining was typically reduced significantly in sections treated with a DBR after staining. There was no significant change in the resolution and fluorescence intensity of the specific nuclear histone staining in brain sections treated with a DBR after staining.

### Example 7

### Additional Polymeric Background Reducing Materials

Any number of polymeric materials that have multiple sulfate, sulfonate, phosphate and/or phosphonate groups may be used in the disclosed compositions, methods and kits. Exemplary polymeric materials are provided in Table 6 below.

TABLE 6
Additional Polymeric Background Reducing Materials

Synthetic Sulfated Polymers
poly(propenesulfate)
poly(butenesulfate)
poly(pentanesulfate)
poly(hexenesulfate)
poly(heptenesulfate)
poly(octenesulfate)
poly(nonenesulfate)
poly(decenesulfate)
poly(undecenesulfate)
poly(dodecenesulfate)
poly(phenylnonenesulfate)
poly(phenyldecenesulfate)
poly(phenylundecenesulfate)
poly(phenyldodecenesulfate)
poly(styrenesulfate)
poly(vinylnaphthalenesulfate)
poly(vinylbiphenylsulfate)
poly(sulfatephenylpropene)
poly(sulfatephenylbutene)
poly(sulfatephenylpentene)
poly(sulfatephenylhexene)
poly(sulfatephenylheptene)
poly(sulfatephenyloctene)
poly(sulfatephenylnonene)
poly(sulfatephenyldecene)
poly(sulfatephenylundecene)
poly(sulfatephenyldodecene)
Synthetic Sulfonated Polymers
poly(propenesulfonate)
poly(butenesulfonate)
poly(pentenesulfonate)
poly(hexenesulfonate)
poly(heptenesulfonate)
poly(octenesulfonate)
poly(nonenesulfonate)
poly(decenesulfonate)
poly(undecenesulfonate)
poly(dodecenesulfonate)
poly(vinylnaphthalenesulfonate)
poly(vinylbiphenylsulfonate)
sulfonated poly(vinylphenylketone)
sulfonated poly(phenylsulfone)
sulfonated poly(4-methylstyrene)
sulfonated poly(alpha-methylstyrene)
sulfonated poly(styrene-block-ethyleneoxide-block-styrene)
sulfonated poly(ethyleneoxide-block-styrene-block-ethyleneoxide)
sulfonated poly(4-methoxystyrene)
sulfonated poly(ethyleneoxide-block-styrene)
sulfonated poly(styrene-block-ethylene)
sulfonated poly(acenaphthylene)
sulfonated poly(vinylcarbazole)
sulfonated poly(styrene-co-butadiene)
sulfonated poly(styrene-block-(ethylene-co-butylene)-block-styrene
poly(naphthalene-2-sulfonate)
poly(methylenehydroquinonesulfonate)
poly(styrenesulfonate-co-styrene)
poly(styrenesulfonate-co-acrylic acid)
poly(styrenesulfonate-co-methacrylic acid)
poly(styrenesulfonate-co-acrylamidomethylpropanesulfonate)
poly(styrenesulfonate-co-itaconic acid)
poly(styrenesulfonate-co-vinylbenzoic acid)
poly(styrenesulfonate-co-octylstyrenesulfonamide)
polystyrenesulfonate-co-menthylstyrenesulfonate)
poly(styrenesulfonate-co-lithocholic acid styrenesulfonate)
poly(styrenesulfonate-co-diallylmethylammonium chloride)
poly(styrenesulfonate-co-diallyldimethylammonium chloride)
poly(styrenesulfonate-co-diallylmethyloctylammonium chloride)
poly(styrenesulfonate-co-allylamine)
poly(styrenesulfonate-co-vinylamine)
poly(styrenesulfonate-co-vinylbenzyltrimethylammonium chloride)
Other Synthetic Sulphur-Containing Polymers
poly(sulfophenethylacrylamide)
poly(sulfophenethylmethacrylamide)
poly(sulfophenylpropene)
poly(sulfophenylbutene)
poly(sulfophenylpentene)
poly(sulfophenylhexene)
poly(sulfophenylheptene)
poly(sulfophenyloctene)
poly(sulfophenylnonene)
poly(sulfophenyldecene)
poly(sulfophenylundecene)
poly(sulfophenyldodecene)
poly(styrenesulfanilate)
Synthetic Phosphate-Containing Polymers
poly(propenephosphate)
poly(butenephosphate)
poly(pentenephosphate)
poly(hexenephosphate)
poly(heptenephosphate)
poly(octenephosphate)
poly(nonenephosphate)
poly(decenephosphate)
poly(undecenephosphate)
poly(dodecenephosphate)
poly(propenephosphonate)
poly(butenephosphonate)
poly(pentenephosphonate)
poly(hexenephosphonate)
poly(heptenephosphonate)
poly(octenephosphonate)
poly(nonenephosphonate)
poly(decenephosphonate)
poly(undecenephosphonate)
polydodecenephosphonate)
poly(phosphophenylpropene)
poly(phosphophenylbutene)
poly(phosphophenylpentene)
poly(phosphophenylhexene)
poly(phosphophenylheptene)
poly(phosphophenyloctene)
poly(phosphophenylnonene)
poly(phosphophenyldecene)
poly(phosphophenylundecene)
poly(phosphophenyldodecene)
poly(phosphatephenylpropene)
poly(phosphatephenylbutene)
poly(phosphatephenylpentene)
poly(phosphatephenylhexene)
poly(phosphatephenylheptene)
poly(phosphate phenyloctene)
poly(phosphate phenylnonene)
poly(phosphatephenyldecene)
poly(phosphatephenylundecene)
poly(phosphatephenyldodecene)
poly(diphenoxyphosphazene)
Carbohydrate Polymers
carrageenan
pentosan sulfate
pentosan phosphate
pentosan phophosulfate
cellulose sulfate
cellulose phosphate
cellulose phophosulfate
dextrin sulfate
dextrin phosphate
dextrin phosphosulfate
laminarin sulfate
laminarin phosphate
Laminarinphosphosulfate
dermatan sulfate
dermatan phosphate
dermatan phosphosulfate
chitin sulfate
chitin phosphate
chitin phosphosulfate
chitosan sulfate
chitosan phosphate
chitosan phosphosulfate
curdlan sulfate
curdlan phosphate
curdlan phosophosulfate
pullulan sulfate
pullulan phosphate
pullulan phosphosulfate
hyaluronic acid sulfate
hyaluronic acid phosphate
hyaluronic acid phosphosulfate
keratan sulfate
keratan phosphate
keratan phosphosulfate
fucoidan sulfate
fucoidan phosphate
fucoidan phosphosulfate
ficoll sulfate
ficoll phosphate
ficoll phosphosulfate
xylan sulfate
xylan phosphate
xylan phosphosulfate
amylose sulfate
amylose phosphate
amylose phosphosulfate
D-galactan sulfate
D-galactan phosphate
D-galactan phosphosulfate
N-(carboxymethyl)chitosan sulfate
N-(carboxymethyl)chitosan phosphate
N-(carboxymethyl)chitosan phosphosulfate
mucin sulfate
mucin phosphate
mucin phosphosulfate
galactomannan sulfate
galactomannan phosphate
galactomannan phosphosulfate
mannan sulfate
mannan phosphate
mannan phosphosulfate
glucan sulfate
glucan phosphate
glucan phosphosulfate
fucan sulfate
fucan phosphate
fucan phosphosulfate
N-acetylheparosan sulfate
N-acetylheparosan phosphate
N-acetylheparosan phosphosulfate
rhamnan sulfate
rhamnan phosphate
rhamnan phosphosulfate
Small Polymeric
Molecules
(−)-epicatechin sulfate
4-sulfocalix[4]arene
4-sulfonatocalix[8]arene
Sulfated Proteins and Peptides
sulfated insulin
polymeric sulfated IgA
sulfated silk fibroin
gastrin sulfate
cholecystokinin
poly(tyrosinesulfate)
poly(sulfophenylalanine)

### Example 8

### Dye Background Blocking Generally Correlates with Increasing Negative Charge

Although not wishing to be bound by any particular theory, it appears that in some embodiments the effectiveness of the disclosed polymeric materials for reducing background staining by dye-conjugates correlates with the particular total negative charge exhibited by the polymeric material. Table 7 below demonstrates that amongst several sulfonated and sulfated polymeric materials tested, compounds with an average charge of −40 or below, for example, −50 or below, appear to be amongst the most effective materials for reducing background staining. However, a direct correlation between the relative average negative charge and the relative background reducing effectiveness of the compounds is not always demonstrated by the data. For example, while sulfobutylether beta-cyclodextrin has a higher average negative charge than sulfopropyl beta-cyclodextrin, it is less effective under the test conditions for blocking background staining by dye-conjugates than sulfopropyl beta-cyclodextrin. Thus, it appears that the charge properties of the polymeric materials are only partially responsible for the observed background staining reduction.

TABLE 7
Chemical and Charge Properties of Selected Background
Reducing Materials

| | | | |
|---|---|---|---|
| | | Average | |
| | SO₄ or SO₃ | Negative | Blocking |
| Compound | Content (%)¹ | Charge³ | Score⁴ |

Linear Polymers

| | | | |
|---|---|---|---|
| heparin MW = 12,000 | ˜20.0² | −75.0 | +++ |
| dextran sulfate MW = 45,000 | 19.4 | −72.8 | +++ |
| poly(styrenesulfonic acid) | 12.9 | −48.4 | +++ |
| MW = 70,000 | | | |
| poly(anilinesulfonic acid) | 12.5 | −43.4 | +++ |
| MW = 10,000 | | | |
| poly(anetholesulfonic acid) | 11.7 | −43.9 | +++ |
| MW = 10,000 | | | |
| poly(2-acrylamido-2-methyl-1- | 14.0 | −52.5 | +++ |
| propanesulfonic acid) | | | |
| MW = 2,000,000 | | | |
| chondroitin sulfate MW = 10,000 | 7.7 | −29.0 | ++ |
| heparin MW = 5,060 | 11.0 | −41.3 | ++ |
| suramin MW = 1,429 | 13.0 | −48.8 | ++ |
| poly[di(ethyleneglycol)/ | 6.5 | −24.4 | — |
| cyclohexanedimethanol-alt- | | | |
| isophthalic acid, sulfonated] | | | |
| MW = 8,000 | | | |
| heparan MW = 30,000 | 6.0 | −22.5 | — |
| poly(ethyleneglycol)-4- | 2.3 | −8.6 | — |
| nonaphenyl-3-sulfopropyl | | | |
| ether MW = 1,261 | | | |

Cyclic Polymers

| | | | |
|---|---|---|---|
| Tetradeca sulfated beta- | 15.7 | −58.9 | +++ |
| cyclodextrin | | | |
| sulfopropyl beta-cyclodextrin | 4.5 | −16.9 | + |
| sulfobutylether beta-cyclodextrin | 9.6 | −36.0 | — |

¹data provided by compound vendor
²data from Jaques, Meth. Biochem. Anal. 24: 203-312 (1977)
³value for heparin (MW = 12,000) from Linhardt, J. Med. Chem., 46: 2551-2564 (2003); values for others computed based on their SO₄/SO₃ content
⁴data from Table 1

Examination of data presented in Table 5 above also reveals that the average molecular weight of the polymeric material also may be a factor in the effectiveness of a particular polymeric material as a DBR. For example, the largest poly(sodium-4-styrene sulfonic acid) material that was tested had an average molecular weight of 1,000,000 and was less effective than two smaller versions of this polymer having average molecular weights of 200,000 and 70,000, respectively. A similar apparent relationship between polymer size and background reducing ability was observed with the dextran sulfates tested. The dextran sulfate with an average molecular weight of 40,000-50,000 was a more effective DBR than both a smaller (<15,000) and a larger (500,000) version of this material. Moreover, the two smaller heparins tested, with average molecular weights of about 3,000 and about 5,000, were less effective than native heparin, which has an average molecular weight of 12,000 or greater. Although not wishing to be bound by any particular theory, it appears that DBR ability is related, at least in part, to a combination of a polymeric material's relative negative charge and its average size.

### Example 9

### Quantitation of Reductions in Non-Specific Nuclear Background Staining Using Heparin

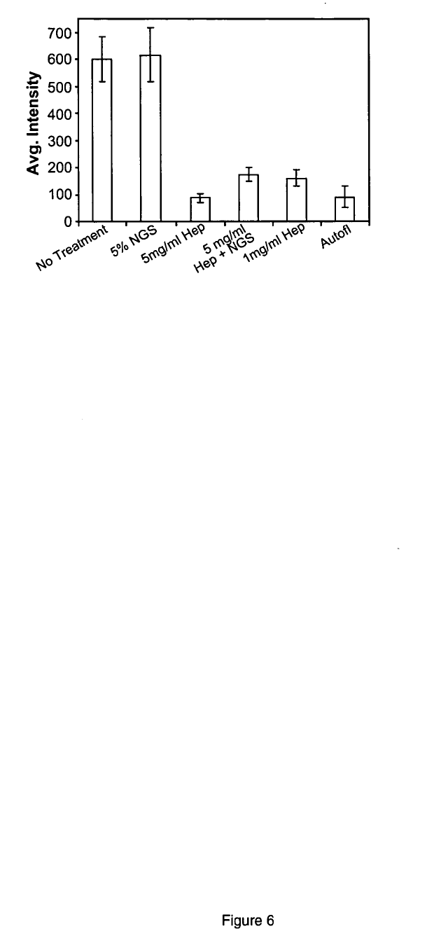

Cultured bovine pulmonary arterial endothelial cells placed on coverslips were fixed in 4% formaldehyde and then their membranes were permeabilized with 0.2% Triton X-100. Different samples of these cells were separately treated with 5% normal goat serum (NGS), 5 mg/mL heparin, 5 mg/mL heparin+5% NGS, or 1 mg/mL heparin. An untreated sample was also prepared as a control. The control sample and the treated samples were then labeled with 5 μg/ml fluorescent (Alexa Fluor 488 dye) goat anti-rabbit (Molecular Probes, Eugene, Oreg.) secondary antibodies for 2 hours. An additional control sample that was both untreated and unlabeled was used to measure native autofluorescence of the cells. No primary antibody was used (that is, all binding was non-specific). The secondary antibody was washed off, and coverslips were mounted on slides with ProLong antifade mounting medium. Three digital images of each sample were taken with a Nikon E800 fluorescence microscope using the same exposure time, objective, and filter set. Images were analyzed for comparative non-specific binding (=“background”) intensities using MetaMorph analysis software, and graphed using Microsoft Excel, which also was used to calculate an average fluorescence intensity and a standard deviation. As shown in FIG. 6, use of NGS blocking (only) did not reduce the background (615+/−100). Use of 5 mg/mL heparin+NGS, or 1 mg/mL heparin (only) reduced the background 71% (174+/−27) and 73% (160+/−32), respectively. Heparin alone (5 mg/mL) reduced the background by 85.7% (86+/−16), which was not statistically different from native autofluorescence (89+/−39).

### Example 10

### Quantitation of Reductions in Non-Specific White Matter Background Staining Using Heparin

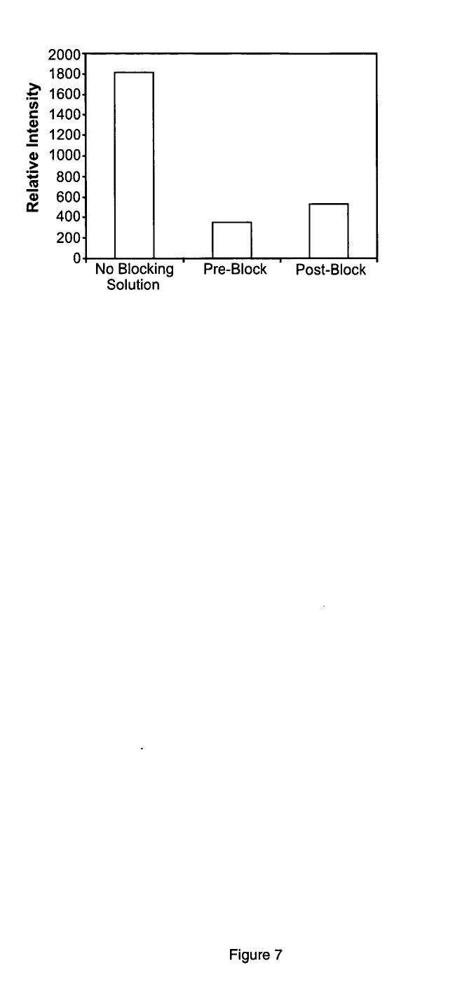

Sixteen μm-thick cryosections of mouse brain hippocampus on microscope slides were rehydrated and permeabilized with 0.2% Triton X-100. Sections were treated for antigen retrieval by placing them in 0.05M TRIS buffer and irradiating them in a microwave for 20 minutes. This increases non-specific binding and is a more rigorous test of a blocking solution's background reducing effectiveness. One section was treated with the present blocking solution before labeling for 2 hours with 5 μg/mL of a fluorescent goat anti-mouse (GAM) secondary antibody (Alexa Fluor 488 GAM, Molecular Probes, Eugene, Oreg.), one section was treated with a present blocking solution after labeling with the fluorescent secondary antibody, and one section was never treated (but was also labeled). No primary antibody was used (that is, all binding was non-specific). Solutions were washed off, and the sections were mounted under coverslips with ProLong antifade solution. After the mountant had hardened, sections were imaged using a Nikon E800 fluorescence microscope using the same filter set, exposure time, and objective. MetaMorph image analysis software was used to determine average intensity, and Microsoft Excel was used to compare intensities. As shown in FIG. 7, Blocking prior to labeling (=“pre-block”) reduced white matter (mainly myelin) intensity down to approximately the level of autofluorescence, a decrease of 80.5% (352.61) from the no-block control (1811.83). Use of the present blocking solution after labeling (=“post-block”) also reduced the intensity by 71.01% (525.31).

### Example 11

### Surface Plasmon Resonance Spectroscopic Assessment of Inhibition of Dye Background Staining by a DBR Solution Applied Before Staining

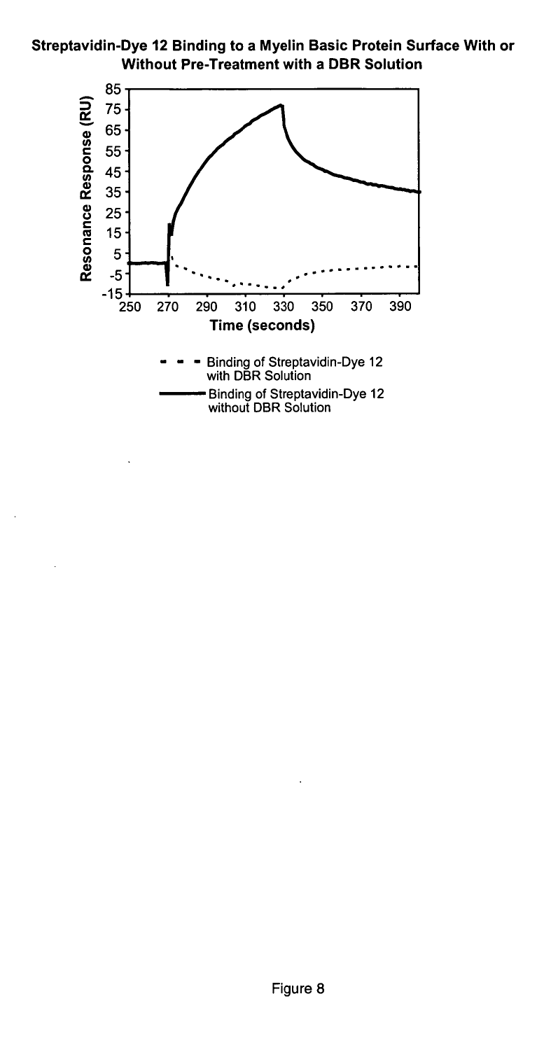

Histone proteins and myelin basic protein are two highly positively-charged constituents characteristic of cell nuclei and white matter, respectively. Because of their high density of positive charges, these proteins may be responsible for at least some of the non-specific staining of nuclei and white matter observed with conjugates containing negatively-charged fluorescent dyes. Such non-specific staining is inhibited by either pre- or post-staining application of a DBR solution to the sample (see FIGS. 1-7). The binding interactions of representative streptavidin conjugates containing negatively-charged fluorescent dyes with a histone protein (calf thymus, H9250, Sigma/Aldrich, Saint Louis, Mo.) and myelin basic protein (rabbit brain, M2016, Sigma/Aldrich) were assessed by surface plasmon resonance (SPR) spectroscopy using a Biacore 3000 SPR spectrometer (Biacore Inc., Piscataway, N.J.). Biacore sensor chips with the standard CM5 carboxymethyl dextran surface were used and the proteins were covalently attached to separate flow cells within the sensor chips according to protocols supplied by the manufacturer. Selected streptavidin-negatively-charged fluorescent dye conjugates were applied to the protein-derivatized surfaces and the resulting SPR signals, or lack thereof, indicating non-specific binding or not, respectively, to the protein-coated surfaces were acquired. It should be noted that binding in these experiments is designated as non-specific because none of the immobilized proteins or unoccupied sensor chip surfaces contained any biotin, the specific ligand that binds very tightly to streptavidin. To assure that only non-specific binding interactions were measured, all streptavidin-fluorescent dye conjugates were pre-saturated with biotin before use. Streptavidin conjugates containing Dyes 12, 14, 21, and 22 (see Table 3) all bound non-specifically to both histone and myelin basic protein-coated surfaces, as indicated by an increase in the SPR signal over background. Binding of one or more components of the DBR solution to the protein-coated surfaces was also detected, but the resulting SPR signals were negligible compared to those obtained with the negatively-charged dye conjugates. In some experiments, the sensor surfaces were pre-treated with a DBR solution immediately before application of the streptavidin-dye conjugate. Pre-treatment of both protein-coated surfaces with a representative DBR solution (see Example 2) totally inhibited non-specific binding of the four fluorescent dye-streptavidin conjugates. Representative SPR spectra illustrating this for streptavidin-dye 12 on a myelin basic protein surface are shown in FIG. 8. The inhibition of non-specific staining of representative positively-charged protein constituents of cell nuclei and white matter immobilized to a synthetic biosensor surface by a DBR solution is qualitatively similar to what is observed in actual cell nuclei and white matter when they are pre-treated with the same DBR solution and then incubated with the same streptavidin-dye conjugates. These data are also consistent with the hypothesis that neutralization of endogenous positive charges by negatively-charged components of a DBR solution is responsible, at least in part, for its ability to block background staining.

The preceding examples can be repeated with similar success by substituting the specifically described compounds of the preceding examples with those generically and specifically described in the foregoing description. One skilled in the art can use the disclosed compositions, methods and kits as specifically described, and without departing from the spirit and scope of the following claims, can make various changes and modifications to adapt to various usages and conditions.

We claim:

1. A blocking solution for reducing background staining, comprising:
a polymeric material comprising multiple carboxylate, sulfate, sulfonate, phosphate, or phosphonate groups, wherein, if the polymeric material is a poly(amino acid), the polymeric material comprises a sulfated, sulfonated or phosphonated poly(amino acid), poly(aspartic acid) or poly(glutamic acid); and
a buffer or water.

2. The blocking solution according to claim 1, wherein the polymeric material has a concentration from about 0.1 mg/mL to about 20 mg/mL.

3. The blocking solution according to claim 1, wherein the polymeric material has a concentration from about 0.5 mg/mL to about 10 mg/mL.

4. The blocking solution according to claim 1, wherein the polymeric material has a concentration from about 1 mg/mL to about 5 mg/mL.

5. The blocking solution according to claim 1, wherein the buffer does not substantially interfere with a specific binding reaction of a dye-conjugate.

6. The blocking solution according to claim 1, wherein the buffer comprises a phosphate (PB), Tris, carbonate, bicarbonate, borate, citrate, acetate, BES, Bicine, CAPS, EPPS, HEPES, MES, MOPS, PIPES, TAPS, TES, Tricine, trimethylammonium acetate, ADA, ACES, MOPSO, TAPSO, DIPSO, AMPD, AMPSO, CAPSO or phosphate buffered saline (PBS) buffer.

7. The blocking solution according to claim 1, wherein the buffer comprises a PBS buffer.

8. The blocking solution according to claim 1, further comprising a detergent.

9. The blocking solution according to claim 1, further comprising a preservative.

10. The blocking solution according to claim 1, further including a dye-conjugate.

11. The blocking solution according to claim 1, wherein the polymeric material comprises a synthetic polymer, a nucleic acid polymer, a carbohydrate polymer or a sulfated, sulfonated, or phosphonated poly(amino acid), or a combination or mixture thereof.

12. The blocking solution according to claim 1, wherein the polymeric material comprises a sulfated or sulfonated carbohydrate.

13. The blocking solution according to claim 1, wherein the polymeric material comprises a polystryrene or copolymer thereof; a polyacrylamide or copolymer thereof; a polyvinylene or copolymer thereof; a polyacrylate or copolymer thereof; a polyalkalene or copolymer thereof; a polyaniline or copolymer thereof; a polyphenylalkylene or copolymer thereof; a glycosaminoglycan or derivative thereof; a heparin or derivative thereof; a dextran or derivative thereof; a suramin or derivative thereof; carrageenan or a derivative thereof; a cyclodextrin other than sulfobutylether beta-cyclodextrin or derivative thereof; a cellulose or derivative thereof; a pentosan or derivative thereof; a dextrin or derivative thereof; a laminarin or derivative thereof; a dermatan or derivative thereof; a chitin or derivative thereof; a chitosan or derivative thereof; a curdlan or derivative thereof; a pullulan or derivative thereof; a keratan or derivative thereof; a fucoidan or derivative thereof; a ficoll or derivative thereof, a xylan or derivative thereof; an amylose or derivative thereof; a galactan or derivative thereof; a mucin or derivative thereof; a galactomannan or derivative thereof; a mannan or derivative thereof; a glucan or derivative thereof; a fucan or derivative thereof; a heparaosan or derivative thereof; a rhamnan or derivative thereof; a catechin or derivative thereof; or, a calixarene or derivative thereof.

14. The blocking solution according to claim 1, wherein the polymeric material comprises poly(sodium 4-styrenesulfonic acid), poly(4-styrenesulfonic acid-co-maleic acid), poly(2-acrylamido-2-methyl-1-propanesulfonic acid), poly(vinylsulfate), poly(vinylsulfonic acid), poly(vinylphosphate), poly(vinylphosphonic acid), poly(anilinesulfonic acid), poly(anetholesulfonic acid), heparin, heparin-like substance, deaminated heparin, chondroitin sulfate, dextran sulfate, sulfopropyl-beta-cyclodextrin, beta-cyclodextrin tetradecasulfate, poly(1-tetradecene-sulfone), poly(ethyleneglycol)-4-nonylphenyl-3-sulfopropyl ether, suramin, poly(propenesulfate), poly(butenesulfate), poly(pentanesulfate), poly(hexenesulfate), poly(heptenesulfate), poly(octenesulfate), poly(nonenesulfate), poly(decenesulfate), poly(undecenesulfate), poly(dodecenesulfate), poly(phenylnonenesulfate), poly(phenyldecenesulfate), poly(phenylundecenesulfate), poly(phenyldodecenesulfate), poly(styrenesulfate), poly(vinylnaphthalenesulfate), poly(vinylbiphenylsulfate), poly(sulfatephenylpropene), poly(sulfatephenylbutene), poly(sulfatephenylpentene), poly(sulfatephenylhexene), poly(sulfatephenylheptene), poly(sulfatephenyloctene), poly(sulfatephenylnonene), poly(sulfatephenyldecene), poly(sulfatephenylundecene), poly(sulfatephenyldodecene), poly(propenesulfonate), poly(butenesulfonate), poly(pentenesulfonate), poly(hexenesulfonate), poly(heptenesulfonate), poly(octenesulfonate), poly(nonenesulfonate), poly(decenesulfonate), poly(undecenesulfonate), poly(dodecenesulfonate), poly(vinylnaphthalenesulfonate), poly(vinylbiphenylsulfonate), sulfonated poly(vinylphenylketone), sulfonated poly(phenylsulfone), sulfonated poly(4-methylstyrene), sulfonated poly(alpha-methylstyrene), sulfonated poly(styrene-block-ethyleneoxide-block-styrene), sulfonated poly(ethyleneoxide-block-styrene-block-ethyleneoxide), sulfonated poly(4-methoxystyrene), sulfonated poly(ethyleneoxide-block-styrene), sulfonated poly(styrene-block-ethylene), sulfonated poly(acenaphthylene), sulfonated poly(vinylcarbazole), sulfonated poly(styrene-co-butadiene), sulfonated poly(styrene-block-(ethylene-co-butylene)-block-styrene, poly(naphthalene-2-sulfonate), poly(methylenehydroquinonesulfonate), poly(styrenesulfonate-co-styrene), poly(styrenesulfonate-co-acrylic acid), poly(styrenesulfonate-co-methacrylic acid), poly(styrenesulfonate-co-acrylamidomethylpropanesulfonate), poly(styrenesulfonate-co-itaconic acid), poly(styrenesulfonate-co-vinylbenzoic acid), poly(styrenesulfonate-co-octylstyrenesulfonamide), polystyrenesulfonate-co-menthylstyrenesulfonate), poly(styrenesulfonate-co-lithocholic acid styrenesulfonate), poly(styrenesulfonate-co-diallylmethylammonium chloride), poly(styrenesulfonate-co-diallyldimethylammonium chloride), poly(styrenesulfonate-co-diallylmethyloctylammonium chloride), poly(styrenesulfonate-co-allylamine), poly(styrenesulfonate-co-vinylamine), poly(styrenesulfonate-co-vinylbenzyltrimethylammonium chloride), poly(sulfophenethylacrylamide), poly(sulfophenethylmethacrylamide), poly(sulfophenylpropene), poly(sulfophenylbutene), poly(sulfophenylpentene), poly(sulfophenylhexene), poly(sulfophenylheptene), poly(sulfophenyloctene), poly(sulfophenylnonene), poly(sulfophenyldecene), poly(sulfophenylundecene), poly(sulfophenyldodecene), poly(styrenesulfanilate), poly(propenephosphate), poly(butenephosphate), poly(pentenephosphate), poly(hexenephosphate), poly(heptenephosphate), poly(octenephosphate), poly(nonenephosphate), poly(decenephosphate), poly(undecenephosphate), poly(dodecenephosphate), poly(propenephosphonate), poly(butenephosphonate), poly(pentenephosphonate), poly(hexenephosphonate), poly(heptenephosphonate), poly(octenephosphonate), poly(nonenephosphonate), poly(decenephosphonate), poly(undecenephosphonate), polydodecenephosphonate), poly(phosphophenylpropene), poly(phosphophenylbutene), poly(phosphophenylpentene), poly(phosphophenylhexene), poly(phosphophenylheptene), poly(phosphophenyloctene), poly(phosphophenylnonene), poly(phosphophenyldecene), poly(phosphophenylundecene), poly(phosphophenyldodecene), poly(phosphatephenylpropene), poly(phosphatephenylbutene), poly(phosphatephenylpentene), poly(phosphatephenylhexene), poly(phosphatephenylheptene), poly(phosphate phenyloctene), poly(phosphate phenylnonene), poly(phosphatephenyldecene), poly(phosphatephenylundecene), poly(phosphatephenyldodecene), poly(diphenoxyphosphazene), carrageenan, pentosan sulfate, pentosan phosphate, pentosan phophosulfate, cellulose sulfate, cellulose phosphate, cellulose phophosulfate, dextrin sulfate, dextrin phosphate, dextrin phosphosulfate, laminarin sulfate, laminarin phosphate, laminarin phosphosulfate, dermatan sulfate, dermatan phosphate, dermatan phosphosulfate, chitin sulfate, chitin phosphate, chitin phosphosulfate, chitosan sulfate, chitosan phosphate, chitosan phosphosulfate, curdlan sulfate, curdlan phosphate, curdlan phosophosulfate, pullulan sulfate, pullulan phosphate, pullulan phosphosulfate, hyaluronic acid sulfate, hyaluronic acid phosphate, hyaluronic acid phosphosulfate, keratan sulfate, keratan phosphate, keratan phosphosulfate, fucoidan sulfate, fucoidan phosphate, fucoidan phosphosulfate, ficoll sulfate, ficoll phosphate, ficoll phosphosulfate, xylan sulfate, xylan phosphate, xylan phosphosulfate, amylose sulfate, amylose phosphate, amylose phosphosulfate, D-galactan sulfate, D-galactan phosphate, D-galactan phosphosulfate, N-(carboxymethyl)chitosan sulfate, N-(carboxymethyl)chitosan phosphate, N-(carboxymethyl)chitosan phosphosulfate, mucin sulfate, mucin phosphate, mucin phosphosulfate, galactomannan sulfate, galactomannan phosphate, galactomannan phosphosulfate, mannan sulfate, mannan phosphate, mannan phosphosulfate, glucan sulfate, glucan phosphate, glucan phosphosulfate, fucan sulfate, fucan phosphate, fucan phosphosulfate, N-acetylheparosan sulfate, N-acetylheparosan phosphate, N-acetylheparosan phosphosulfate, rhamnan sulfate, rhamnan phosphate, rhamnan phosphosulfate, (−)-epicatechin sulfate, 4-sulfocalix[4]arene, 4-sulfonatocalix[8]arene, sulfated insulin, polymeric sulfated IgA, polymeric sulfated IgD, polymeric sulfated IgE, polymeric sulfated IgG, polymeric sulfated IgM, sulfated silk fibroin, gastrin sulfate, cholecystokinin, poly(aspartic acid), poly(glutamic acid), poly(tyrosinesulfate), or poly(sulfophenylalanine), or a combination or mixture thereof.

15. The blocking solution according to claim 1, wherein the polymeric material comprises poly(sodium 4-styrenesulfonic acid), poly(4-styrenesulfonic acid-co-maleic acid), poly(2-acrylamido-2-methyl-1-propanesulfonic acid), poly(vinylsulfate), poly(vinylsulfonic acid), poly(vinylphosphate), poly(vinylphosphonic acid), poly(anilinesulfonic acid), poly(anetholesulfonic acid), heparin, heparin-like substance, deaminated heparin, chondroitin sulfate, dextran sulfate, sulfopropyl-beta-cyclodextrin, or beta-cyclodextrin tetradecasulfate, or a combination or mixture thereof.

16. The blocking solution according to claim 1, wherein the polymeric material comprises heparin or dextran sulfate, or a combination or mixture thereof.

17. The blocking solution according to claim 16, wherein the heparin is porcine Type 1-A heparin.

18. The blocking solution according to claim 16, wherein the dextran sulfate has an average molecular weight of about from 5,000 to about 1,000,000.

19. The blocking solution according to claim 16, wherein the dextran sulfate has an average molecular weight of from about 15,000 to about 100,000.

20. A blocking solution for reducing background staining by dye-conjugates, comprising: a polymeric material dissolved in a phosphate buffered saline at a concentration from about 0.1 mg/mL to about 20 mg/mL, wherein the polymeric material comprises poly(sodium 4-styrenesulfonic acid), poly(4-styrenesulfonic acid-co-maleic acid), poly(2-acrylamido-2-methyl-1-propanesulfonic acid), poly(vinylsulfate), poly(vinylsulfonic acid), poly(vinylphosphate), poly(vinylphosphonic acid), poly(anilinesulfonic acid), poly(anetholesulfonic acid), heparin, heparin-like substance, deaminated heparin, chondroitin sulfate, dextran sulfate, sulfopropyl-beta-cyclodextrin, or beta-cyclodextrin tetradecasulfate, or a combination or mixture thereof.

21. A method for staining a cell or tissue with a dye-conjugate with reduced non-specific background staining by the dye-conjugate, comprising:
a) contacting the cell or tissue with the dye-conjugate to form a contacted sample, wherein the dye-conjugate specifically binds to a particular component of the cell or tissue;
b) contacting the contacted sample with a blocking solution to form a blocked sample, wherein the blocking solution comprises a polymeric material comprising multiple carboxylate, sulfate, sulfonate, phosphate, or phosphonate groups, wherein, if the polymeric material is a poly(amino acid), the polymeric material comprises a sulfated, sulfonated or phosphonated poly(amino acid), poly(aspartic acid) or poly(glutamic acid); and a buffer or water;
c) incubating the blocked sample for a sufficient amount of time to form an incubated sample, wherein the dye-conjugate specifically binds to a particular component of the cell or tissue and the blocking solution reduces non-specific background staining;
d) illuminating the incubated sample with an appropriate wavelength to form an illuminated sample;
e) observing the illuminated sample whereby the blocking solution reduces non-specific binding of the dye-conjugate to cell components other than the particular component to which the dye-conjugate specifically binds.

22. A kit for reducing non-specific staining of a cell or tissue by a dye-conjugate, comprising;
a) a blocking solution comprising a polymeric material and a buffer or water, wherein the polymeric material comprises multiple carboxylate, sulfate, sulfonate, phosphate, or phosphonate groups, wherein, if the polymeric material is a poly(amino acid), the polymeric material comprises a sulfated, sulfonated or phosphonated poly(amino acid), poly(aspartic acid) or poly(glutamic acid); and
b) instructions explaining how to use the polymeric material to reduce non-specific staining of the cell or tissue by the dye-conjugate.
